(19) United States
(12) Patent Application Publication
(10) Pub. No.: US 2009/0156958 A1
(43) Pub. Date: Jun. 18, 2009

(54) DEVICES AND METHODS FOR PERCUTANEOUS ENERGY DELIVERY

(76) Inventors: Bankim H. MEHTA, San Ramon, CA (US); John E. ASHLEY, Danville, CA (US); Scott A. McGILL, San Ramon, CA (US)

Correspondence Address: LEVINE BAGADE HAN LLP, 2483 EAST BAYSHORE ROAD, SUITE 100, PALO ALTO, CA, 94303, US

(21) Appl. No.: 12/055,258

(22) Filed: Mar. 25, 2008

Related U.S. Application Data
(60) Provisional application No. 61/013,182, filed on Dec. 12, 2007.

(51) Int. Cl. A61F 7/12; A61N 1/05; A61N 7/00; A61B 5/01; A61N 5/06

(52) U.S. Cl. 600/549; 607/2; 607/88; 607/96; 601/2

(57) ABSTRACT

The invention provides a system and method for percutaneous energy delivery in an effective, manner using one or more probes. Additional variations of the system include array of probes configured to minimize the energy required to produce the desired effect.

### CROSS-REFERENCE

This application is a non-provisional of U.S. Provisional Application No. 61/013,182 filed on Dec. 12, 2007 entitled “PERCUTANEOUS ENERGY DELIVERY” the entirety of which is incorporated reference.

### BACKGROUND OF THE INVENTION

The systems and method discussed herein treat tissue in the human body. In a particular variation, systems and methods described below treat cosmetic conditions affecting the skin of various body parts, including face, neck, and other areas traditionally prone to wrinkling, lines, sagging and other distortions of the skin.

Exposure of the skin to environmental forces can, over time, cause the skin to sag, wrinkle, form lines, or develop other undesirable distortions. Even normal contraction of facial and neck muscles, e.g. by frowning or squinting, can also over time form furrows or bands in the face and neck region. These and other effects of the normal aging process can present an aesthetically unpleasing cosmetic appearance.

Accordingly, there is well known demand for cosmetic procedures to reduce the visible effects of such skin distortions. There remains a large demand for “tightening” skin to remove sags and wrinkles especially in the regions of the face and neck.

One method surgically resurfaces facial skin by ablating the outer layer of the skin (from 200 μm to 600 μm), using laser or chemicals. In time, a new skin surface develops. The laser and chemicals used to resurface the skin also irritate or heat the collagen tissue present in the dermis. When irritated or heated in prescribed ways, the collagen tissue partially dissociates and, in doing so, shrinks. The shrinkage of collagen also leads to a desirable “tightened” look. Still, laser or chemical resurfacing leads to prolonged redness of the skin, infection risk, increased or decreased pigmentation, and scarring.

Lax et al. U.S. Pat. No. 5,458,596 describes the use of radio frequency energy to shrink collagen tissue. This cosmetically beneficial effect can be achieved in facial and neck areas of the body in a minimally intrusive manner, without requiring the surgical removal of the outer layers of skin and the attendant problems just listed.

Utely et al. U.S. Pat. No. 6,277,116 also teaches a system for shrinking collagen for cosmetically beneficial purposes by using an electrode array configuration.

However, areas of improvement remain with the previously known systems. In one example, fabrication of an electrode array may cause undesired cross-current paths forming between adjacent electrodes resulting in an increase in the amount of energy applied to tissue.

Thermage, Inc. of Hayward Calif. also holds patents and sells devices for systems for capacitive coupling of electrodes to deliver a controlled amount of radiofrequency energy. This controlled delivery of RF energy creates an electric field through the epidermis that generates “resistive heating” in the skin to produce cosmetic effects while simultaneously attempting to cool the epidermis with a second energy source to prevent external burning of the epidermis.

In such systems that treat in a non-invasive manner, generation of energy to produce a result at the dermis results in unwanted energy passing to the epidermis. Accordingly, excessive energy production creates the risk of unwanted collateral damage to the skin.

In view of the above, there remains a need for an improved energy delivery system. Such systems may be designed to create an improved electrode array delivery system for cosmetic treatment of tissue. In particular, such an electrode array may provide deep uniform heating by applying energy to tissue below the epidermis to cause deep structures in the skin to immediately tighten. Over time, new and remodeled collagen may further produce a tightening of the skin, resulting in a desirable visual appearance at the skin's surface. Such systems can also provide features that increase the likelihood that the energy treatment will be applied to the desired target region. Moreover, devices and systems having disposable or replaceable energy transfer elements provide systems that offer flexibility in delivering customized treatment based on the intended target tissue.

Moreover, the features and principles used to improve these energy delivery systems can be applied to other areas, whether cosmetic applications outside of reduction of skin distortions or other medical applications.

### SUMMARY OF THE INVENTION

The invention provides improved systems and methods of percutaneously delivering energy to tissue. In one aspect of the invention, the methods and systems produce cosmetically beneficial effects of using energy to shrink collagen tissue in the dermis in an effective manner that prevents the energy from affecting the outer layer of skin. However, the devices and method described herein can target the underlying layer of adipose tissue or fat for lipolysis or the breakdown of fat cells. Selecting probes having sufficient length to reach the subcutaneous fat layer allows for such probes to apply energy in the subcutaneous fat layer. Application of the energy can break down the fat cells in that layer allowing the body to absorb the resulting free fatty acids into the blood stream. Such a process can allow for contouring of the body surface for improved appearance. Naturally, such an approach can be used in the reduction of cellulite. In addition, the systems and methods are also useful for treating other skin surface imperfections and blemishes by application of a percutaneous treatment.

The invention includes methods for applying energy treating to a region of tissue beneath the epidermis to produce a therapeutic affect. By selectively applying energy percutaneously rather than through the epidermis, the amount of energy can be significantly reduced thereby avoiding collateral damage to tissue.

The methods include positioning at least a portion of at least one probe beneath the epidermis, where the probe comprises a body having an outer perimeter, and applying energy from the probe to create a zone of treatment, such that the exposure of energy to tissue is non-uniform about the outer perimeter of the probe and greatest in the zone of treatment.

One or more of the probes can be configured to produce any number of zones of treatment. For example, a probe can be configured to have a number of zones along a length of the probe where the amount or intensity of energy at each zone is specific to the region of target tissue. In addition, the probe can be configured to produce zones that combine with adjacent probes to create a treatment size in the intersection of zones between adjacent probes.

As noted above, the method can include an amount of energy to cause a therapeutic effect only in tissue within the zone of treatment. As such, the amount of energy will not be uniform about the perimeter of the probe.

The probes can employ a variety of energy types. For example, the probes can employ energy delivery element such as acoustic transducers, illumination sources, microwave energy supplies, resistive heat sources, RF energy electrodes, as well as a cooling source. As noted herein, variations of the methods and devices include a variety of energy modalities combined in a single probe. Moreover, a variety of energy modalities can be combined in a single array of multiple probes.

As shown herein, the application of energy can be manipulated to redirect the zone of treatment. For example, the energy source can be articulated to change an angular position of the selective direction of energy delivery. Alternatively, or in combination, the energy source or probe can be rotated to selectively apply the energy delivery in numerous directions about the probe.

The systems and methods also include the use of various temperature measuring devices to monitor temperature above and/or beneath the epidermis and adjacent to the treatment site. In some variations, the temperature measuring device can be advanced into the zone of treatment and/or into a path of the energy being applied to the tissue.

The invention also includes devices for percutaneous delivery energy from a power supply to tissue. Such devices can include a body having at least one probe extending therefrom, where the probe has a tip adapted to penetrate tissue, and where a sidewall of the probe comprises an opening allowing for an energy delivery element coupleable to the power supply and positioned within the probe to transmit energy through the opening of the sidewall to treat tissue. In some additional variations, an opening in a probe wall is not required to provide treatment to the tissue. Moreover, the probe may also include shielding or insulation on certain areas so that the application of energy can be directed as needed.

In some variations the devices include a tissue engaging surface on the body where the tissue engaging surface assists in uniform placement of the probe beneath a surface of the tissue.

As noted above, the devices can employ a variety of energy delivery modalities (including, but not limited to acoustic transducers, illumination sources, microwave energy conductors, resistive heat source, an RF energy probe, or a cooling source).

The devices can also optionally include one or more temperature sensing elements located on a probe or on a body of the device. In some variations, the temperature sensing element can be advanced from the probe or device and into the region of tissue being treated.

The devices and methods described herein may provide probe arrays provided in a cartridge body that is removably coupled to a treatment device, where a probe array of the cartridge device can penetrate tissue at an oblique angle or at a normal angle as discussed below. In addition, in those variations where the probe array enters at an oblique angle, the device may include a cooling surface that directly cools the surface area of tissue adjacent to the treated region of tissue. The cooling methods and apparatus described herein may be implemented regardless of whether the probes penetrate at an oblique angle or not.

In one variation of the device, the device comprises: a device body having a handle portion, a cartridge receiving surface, an actuator adjacent thereto and a plurality of electrically conductive leads on at least a portion of the cartridge receiving surface and being electrically coupleable to the energy source, where the actuator is moveable relative to the device body; a cartridge body removably coupled to the device body on the cartridge receiving surface, the cartridge body comprising a probe assembly in engagement with the actuator, the probe assembly having a plurality of probes arranged in an array and at least one of the probes having a connection portion, the probe assembly being moveable between a treatment position and a retracted position upon movement of the actuator, such that in the treatment position one or more probes can extend from the cartridge body and the respective connection portion engages one electrically conductive lead, and in the retracted position, one or more probe retracts into the cartridge and the respective connection portion moves out of engagement with the electrically conductive lead preventing delivery of energy.

In additional variations, the cooling surface pre-cools the skin and underlying epidermis prior to delivering the therapeutic treatment. Additional variations include application of cooling during and/or subsequent to the energy delivery where such cooling is intended to minimize undesired damage to the epidermis, to maintain the epidermis temperature, and/or to retain the epidermis in a normal condition.

Variations of the invention include movement of the probes by use of a spring or other means to provide an impact force to the probes to penetrate tissue. The spring provides a spring force to move the probes at a velocity that allows for easier insertion of the probe array into tissue.

Alternatively, or in combination, the probes may be coupled to an additional source of energy that imparts vibration in the probes (e.g., an ultrasound energy generator). The same energy source may be used to generate the thermal effect in the dermis.

The methods and devices described herein may also use features to facilitate entry of the probes into tissue. For example, the surface tissue may be placed in traction prior to advancing probes through the surface tissue. The probes can comprise a curved shape, where advancing the curved probes through tissue can comprise rotating the probes into tissue.

Another variation of the invention includes a cartridge and/or hand unit having any number of electronic storage units or memory (e.g., SRAM, DRAM, Masked ROM, PROM, EPROM, EEPROM, Flash memory, NVRAM, etc. or any combination thereof). Such memory capabilities can contain instructions or record communication between the cartridge and hand unit and/or controller to adjust treatment parameters, monitor usage, monitor sterility, or to record and convey other system or patient characteristics. In yet another variation, the cartridge and/or hand unit can include an RFID antenna/receiver configuration for preventing or permitting treatment given that the hand unit/controller recognizes a code embedded with the RFID antenna.

It is expressly intended that, wherever possible, the invention includes combinations of aspects of the various embodiments described herein or even combinations of the embodiments themselves.

In addition, the concepts disclosed herein can be combined with the following commonly assigned applications where such combinations are possible: U.S. patent application Ser. No. 11/676,230 entitled “METHODS AND DEVICES FOR TREATING TISSUE filed on Feb. 16, 2007; PCT application No.: PCT/US2007/081556 entitled “METHODS AND DEVICES FOR TREATING TISSUE filed on Oct. 16, 2007; U.S. patent application Ser. No. 11/764,032 entitled “METHODS AND DEVICES FOR TREATING TISSUE filed on Jun. 15, 2007; and U.S. patent application Ser. No. 11/832,544 entitled “METHODS AND DEVICES FOR TREATING TISSUE filed on Aug. 1, 2007. Each of which is incorporated by reference herein.

### BRIEF DESCRIPTION OF THE DRAWINGS

FIG. 1 shows a representative cross sectional view of the skin composed of an outer stratum corneum covering the epidermal and dermal layers of skin and the underlying subcutaneous tissue;

FIG. 2A shows a sample variation of a system according to the principles of the invention having probes configured to provide percutaneous energy delivery;

FIG. 2B illustrates a partial view of a working end of a treatment unit engaging tissue such that the probes enters the tissue;

FIG. 2C shows another variation of a system having probes configured to apply percutaneous energy delivery;

FIGS. 3A to 3B show variations of probes for use with the systems and methods described herein to create a zone of treatment;

FIGS. 4A to 4C show variations of probes for use with an illumination energy source and where the energy source delivery can be articulated with respect to the probe for re-directing a zone of treatment;

FIGS. 5A to 5B show a variation of a probe to move the zone of treatment around the probe to increase a treatment area;

FIGS. 6A to 6E depict various probe array configurations for use in variations of the systems and methods described herein;

FIG. 7 shows a variation of a fluid delivery probe;

FIG. 8 shows a probe having a combination of treatment modalities;

FIG. 9A illustrates a perspective view of a variation of a cartridge body for use with the present system;

FIGS. 9C to 9D show a perspective, side, and top views respectively of an alternate cartridge body for use with the present system;

FIG. 10 shows a graph representing pulsed energy delivery and temperature measurements between pulses of energy;

FIGS. 11A to 11B show variations of introducer members that assist in placing probes within tissue;

FIG. 12A shows an additional variation of a device having an array of probes in a removable cartridge adjacent to a tissue engaging surface;

FIG. 12B shows a magnified view of the probes and tissue engaging surface of the device of FIG. 12A;

FIG. 12C shows an example of an probe entering tissue at an oblique angle adjacent to a tissue engaging surface;

FIG. 13 shows another example of an probe entering tissue at an oblique angle underneath a skin anomaly;

FIG. 14A to 14C show cooling surfaces adjacent to the probes; and

FIGS. 15A to 15D illustrate additional variations of probe for use with the systems and devices described herein.

### DESCRIPTION OF THE PREFERRED EMBODIMENT

The systems and method discussed herein treat tissue in the human body. In one variation, the systems and methods treat cosmetic conditions affecting the skin of various body parts, including face, neck, and other areas traditionally prone to wrinkling, lines, sagging and other distortions of the skin. The methods and systems described herein may also have application in other surgical fields apart from cosmetic applications.

The inventive device and methods also include treatment of skin anomalies such as warts (Verruca plana, Verruca vulgaris), sebaceous hyperplasia or acne (Acne vulgaris). Treatment of acne can be accomplished by the direct ablation of sebaceous glands or it can be accomplished by the delivery of thermal energy which will stimulate the body's immune system to eliminate the bacteria, Propionibacterium acnes, which is one of the causes of acne. The methods and devices can be used for the removal of unwanted hair (i.e., depilation) by applying energy or heat to permanently damage hair follicles thereby removing the skins ability to grow hair. Such treatment may be applied on areas of facial skin as well as other areas of the body.

Other possible uses include pain management (both in the use of heat to reduce pain in muscle tissue and by directly ablating nociceptive pain fibers), stimulation of cellular healing cascade via heat, treatment of the superficial muscular aponeurotic system (SMAS), reproductive control by elevated heating of the testicles, and body modification such as piercing, scarification or tattoo removal

In addition to therapeutic surface treatments of the skin, the current invention can be targeted to the underlying layer of adipose tissue or fat for lipolysis or the breakdown of fat cells. Selecting probes having sufficient length to reach the subcutaneous fat layer allows for such probes to apply energy in the subcutaneous fat layer. Application of the energy can break down the fat cells in that layer allowing the body to absorb the resulting free fatty acids into the blood stream. Such a process can allow for contouring of the body surface for improved appearance. Naturally, such an approach can be used in the reduction of cellulite.

Other possible uses include pain management (both in the use of heat to reduce pain in muscle tissue and by directly ablating nociceptive pain fibers), stimulation of cellular healing cascade via heat, reproductive control by elevated heating of the testicles, and body modification such as scarification.

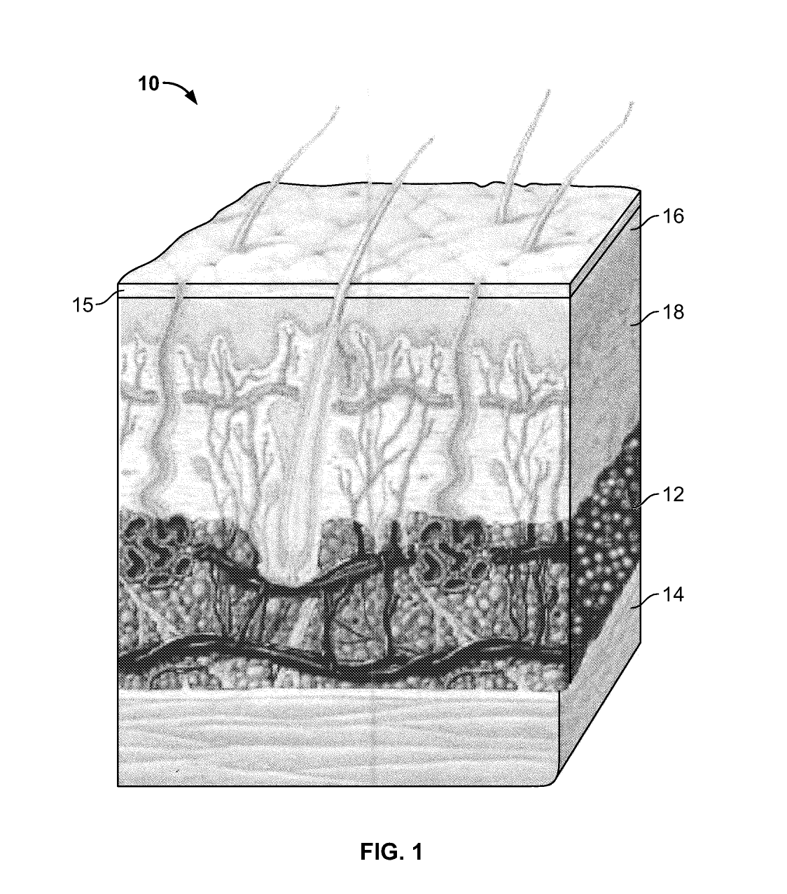

FIG. 1 shows a cross sectional view of the skin 10 composed of an outer stratum corneum 15 covering the epidermis 16. The skin also includes the dermis 18, subcutaneous tissue/fat 12. These layers cover muscle tissue 14 of within the body. In the face and neck areas, the skin 10 measures about 2 mm in cross sectional depth. In the face and neck regions, the epidermis measures about 100 μm in cross sectional depth. The skin 10 also includes a dermis 18 layer that contains a layer of vascular tissue. In the face and neck regions, the dermis 18 measures about 1900 μm in cross sectional depth.

The dermis 18 includes a papillary (upper) layer and a reticular (lower) layer. Most of the dermis 18 comprises collagen fibers. However, the dermis also includes various hair bulbs, sweat ducts, sebaceous glands and other glands. The subcutaneous tissue 12 region below the dermis 18 contains fat deposits as well as vessels and other tissue.

In most cases, when applying cosmetic treatment to the skin for tightening or removal of wrinkles, it is desirable to deliver energy to the dermis layer rather than the epidermis, the subcutaneous tissue region 12 or the muscle 14 tissue. In fact, delivery of energy to the subcutaneous tissue region 12 or muscle 14 may produce pockets or other voids leading to further visible imperfections in the skin of a patient. Also, delivery of excessive energy to the epidermis can cause burns and/or scars leading to further visible imperfections.

The application of heat to the fibrous collagen structure in the dermis 18 causes the collagen to dissociate and contract along its length. It is believed that such disassociation and contraction occur when the collagen is heated to about 65 degree C. The contraction of collagen tissue causes the dermis 18 to reduce in size, which has an observable tightening effect. As the collagen contracts, wrinkles, lines, and other distortions become less visible. As a result, the outward cosmetic appearance of the skin 10 improves. Furthermore, the eventual wound healing response may further cause additional collagen production. This latter effect may further serve to tighten and bulk up the skin 10.

Thermal energy is not the only method for treating collagen in the dermal layer to effect skin laxity and wrinkles. Mechanical disruption or cooling of tissue can also have a desirable therapeutic effect. As such, the devices and methods described herein are not limited to the percutaneous delivery of thermal energy, but also include the percutaneous delivery of mechanical energy or even reducing temperature of tissues beneath the epidermis (e.g., hypothermia effect on tissue).

The treatment methods and device can also include the use of additives, medicines, bioactive substances, or other substances intended to create a therapeutic effect on their own or augment a therapeutic effect created by any one of the energy modalities discussed herein.

For example, autograph or allograph collagen can be delivered percutaneously to bulk up the dermal layer. Non-collagen fillers such as absorbable and non-absorbable polymers can also be delivered to increase the volume of the dermis and improve the surface appearance of the skin. Saline can be delivered to provide a diffuse path for radio frequency current delivery or to add or remove thermal energy from the target tissue. In addition, anesthetic or numbing agents can be delivered to reduce the patient's sensation of pain from the treatment. Botulinum Toxin type A (Botox®) can also be delivered to the dermis or to the muscular layer below the dermis by further inserting the access probe 32. The delivery of Botox® can temporarily paralyze the underlying musculature allowing for treatment of the target area with no muscle movement to move or disturb the treatment area.

The delivery of the substances described above can occur using the same delivery devices that apply the energy based treatment. Alternatively, or in combination, a physician can administer such substances using a delivery means separate from the treatment devices.

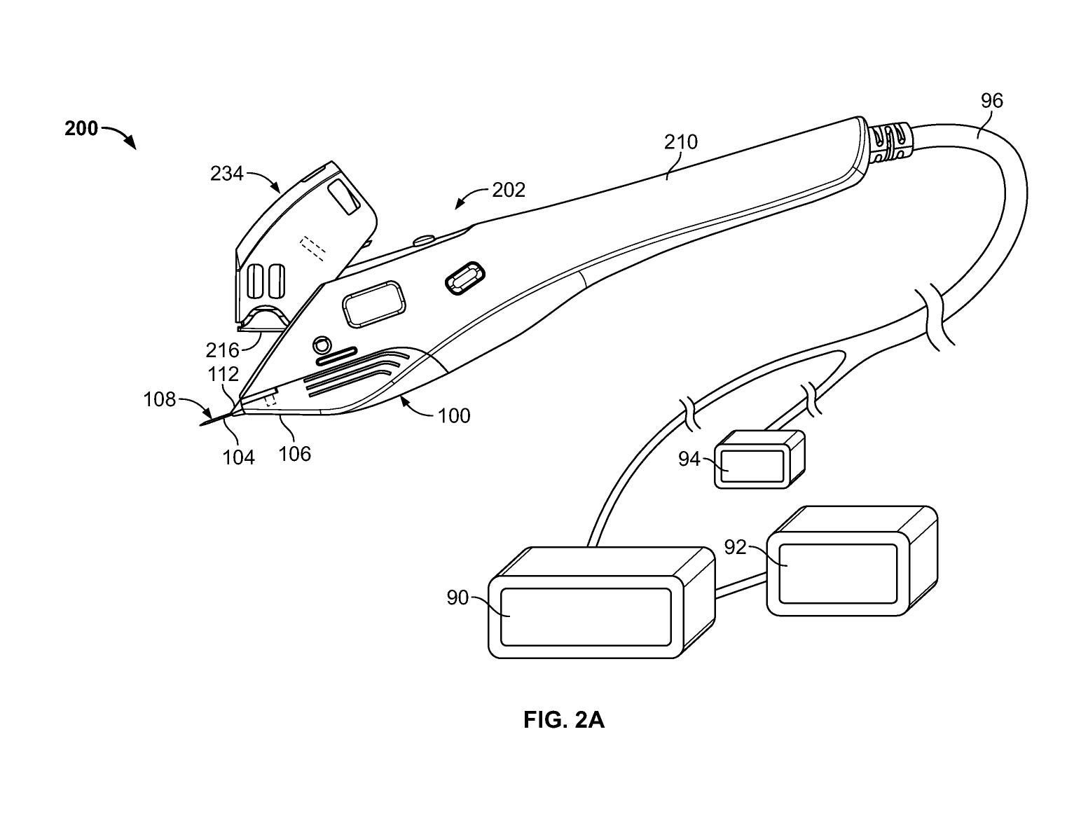

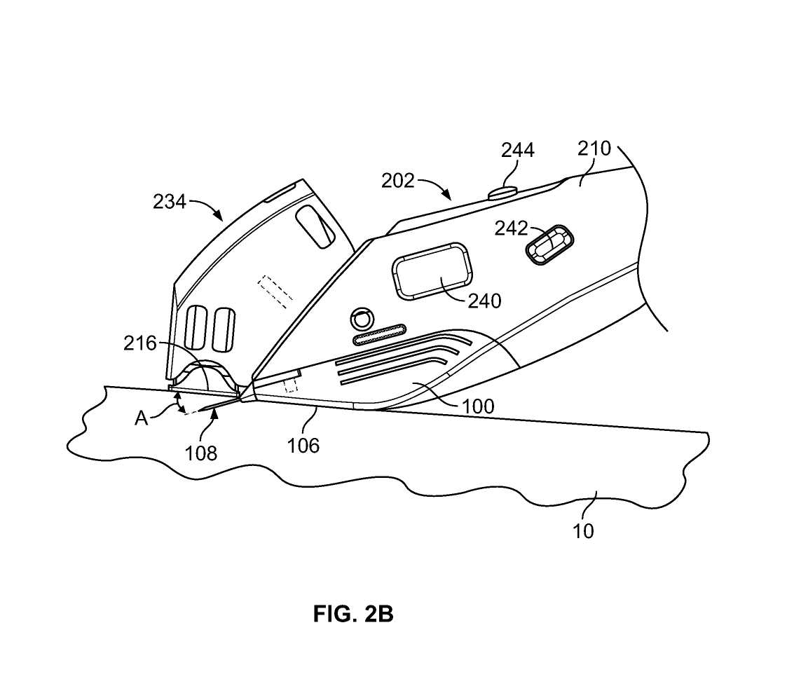

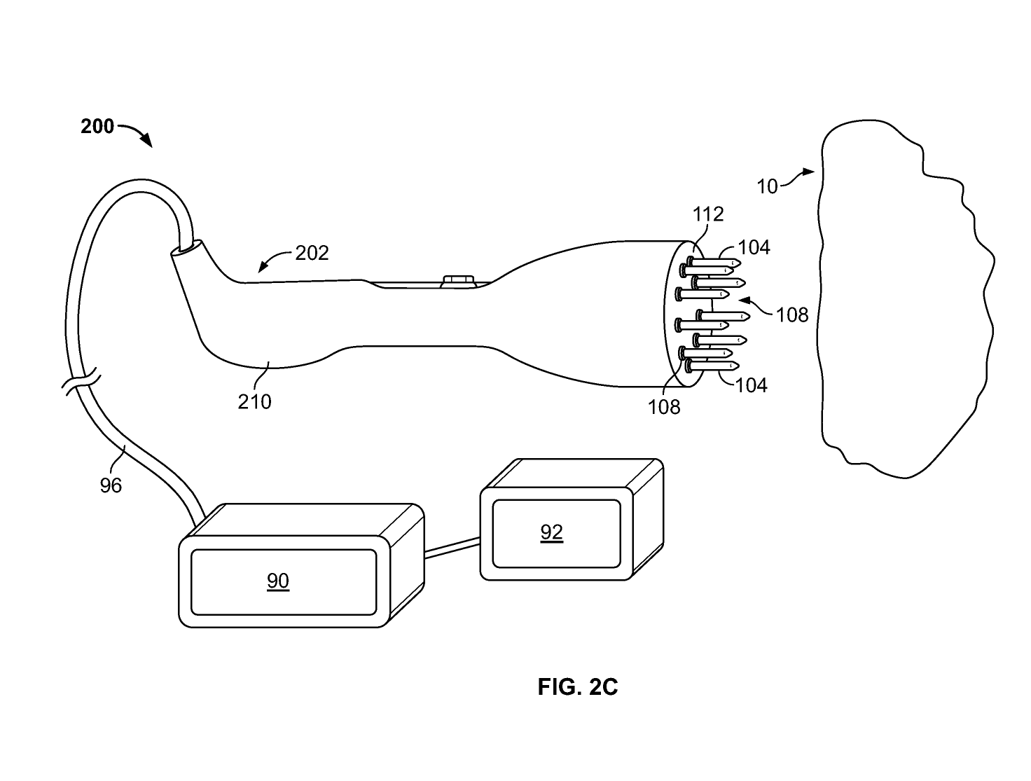

FIG. 2A illustrates one variation of a treatment system according the principles described herein. The treatment system 200 generally includes a treatment unit 202 having a hand-piece or device body 210 (or other member/feature that allows for manipulation of the system to treat tissue 10) having one or more probes 104 extending from the body 210. In some variations, the probes 104 are coupled to the body 210 via a removable cartridge 100. In the system 200 shown, the removable cartridge 100 contains a plurality of retractable probes 104 arranged in an array 108. Hereafter, the term probes 104 is intended to include any electrode, energy transfer element (e.g., thermal, electrical, electromagnetic, microwave, mechanical, ultrasound, etc.), or source of therapeutic treatment. For sake of convenience, the term probe shall be used to refer to any electrode, energy transfer element or source of therapeutic treatment unless specifically noted otherwise. As shown, the probes 104 can optionally extend from a front portion 112 of the cartridge 100. Alternatively, the probes 104 can extend from a front face of the device body or from any surface of the device body/cartridge.

The device body 210 is not limited to that shown. Instead, variations include device body shapes that are thinner in profile and can be held at a more vertical angle to the target tissue like a pencil or pointer. Variations also include a device body that has a loop or curved grip that facilitates one specific manner in which it can be grasped by the hand. Any number of variations is possible especially those that ensure the physician's hand does not contact of the distal end of the cartridge or the target tissue.

The devices according to the principles described herein can include any number of arrays depending upon the intended treatment site. Currently, the size of the array, as well as the number of arrays, can change depending on the variation of the invention needed. In most cases, the target region of tissue drives the array configuration. The present invention allows a physician to selectively change array configuration by attaching different cartridges 100. Alternatively, variations of the invention contemplate an probe assembly that is non-removable from the device body 200.

For example, a treatment unit 202 designed for relatively small treatment areas may only have a single pair of probes. On the other hand, a treatment unit 202 designed for use on the cheek or neck may have up to 10 probe pairs. However, estimates on the size of the probe array are for illustrative purposes only. In addition, the probes on any given array may be the same shape and profile. Alternatively, a single array may have probes of varying shapes, profiles, and/or sizes depending upon the intended application.

Furthermore, the array 108 defined by the individual probes 104 can have any number of shapes or profiles depending on the particular application. As described in additional detail herein, in those variations of the system 200 intended for skin resurfacing, the length of the probes 104 is generally selected so that the energy delivery occurs in the dermis layer of the skin 10 while the spacing of probes 104 may be selected to minimize delivery of energy between adjacent pairs of probes or to minimize energy to certain areas of tissue.

In those variations where the probes 104 are resistive, radiofrequency, microwave, inductive, acoustic, or similar type of energy transfer elements, the probes can be fabricated from any number of materials, e.g., from stainless steel, platinum, and other noble metals, or combinations thereof. Additionally, such probe may be placed on a non-conductive member (such as a polymeric member).

Additionally, the treatment unit 202 may or may not include an actuator as described below for driving the probe array 108 from the cartridge 100 into the target region. Examples of such actuators include, but are not limited to, gas powered cylinders, springs, linear actuators, or other such motors. Alternative variations of the system 200 include actuators driven by the control system/energy supply unit 90.

FIG. 2A also shows an optional cooling device 234 coupled to the device body 210. The cooling device 234 can be adjustable along the device body 210. The use of a cooling device 234 can also be desirable in those cases where energy or heat is applied to the tissue. In addition, a cooling device may have other beneficial effects even when a heat or energy treatment is not being used. In yet additional variations, the cooling device can be replaced with a heating device (such as when a cooling treatment is used to induce the therapeutic treatment within tissue).

In the illustrated variation, the cooling device 234 is in a retracted position where it is spaced away from probes 108 (and thus spaced from the surface of the target tissue). This retracted position can aid the user by allowing for visualization of proper placement of the probe array 108 into the target tissue. After the user places the device 202 on tissue, the user can advance the cooling device 234 (manually or automatically upon activation of the system) so that a cooling surface 216 of the cooling device 234 makes contact with the target tissue.

The cooling device can be an air or liquid type cooling device. Alternatively, the cooling device can include a Peltier cooling device. A Peltier cooling device can eliminate the need for a fluid source. In some cases, the cooling device can be powered using the same power supply that energizes the probes. Such a configuration provides a more compact design that is easier for a medical practitioner to manipulate.

The system 200 also includes an energy supply unit 90 coupled to the treatment unit 202 via a cable 96 or other means. The energy supply unit 90 may contain the software and hardware required to control energy delivery. Alternatively, the CPU, software and other hardware control systems may reside in the hand piece 210 and/or cable 96. It is also noted that the cable 96 may be permanently affixed to the supply unit 90 and/or the treatment unit 202. In additional variations, the hand piece 210 can contain the controls alone or the controls and the power supply necessary to delivery treatment.

In one variation, the energy supply unit 90 may be a RF energy unit. Additional variations of energy supply units may include power supplies to provide or remove thermal energy, to provide ultrasound energy, microwave energy, laser energy, pulsed light energy, and infrared energy. Furthermore, the systems may include combinations of such energy modalities.

For example, in addition to the use of RF energy, other therapeutic methods and devices can be used in combination with RF energy to provide additional or more efficacious treatments. For example, as shown in FIG. 2A, additional energy sources 96 can be delivered via the same or additional energy transfer elements located at the working end of a treatment unit 202. Alternatively, the radiant energy may be supplied by the energy source/supply 90 that is coupled to a diode, fiber, or other emitter at the distal end of the treatment unit 202. In one variation, the energy source/supply 94 and associated energy transfer element may comprise laser, light or other similar types of radiant energy (e.g., visible, ultraviolet, or infrared light). For example, intense pulsed light having a wavelength between 300 and 12000 nm can also be used in conjunction with RF current to heat a targeted tissue. Such associated transfer elements may comprise sources of light at the distal end of the treatment unit 202. These transfer elements may be present on the cartridge 100, on the device body 210 or even on the cooling unity 234. More specifically a coherent light source or laser energy can be used in conjunction with RF to heat a targeted tissue. Examples of lasers that can be used include erbium fiber, CO₂, diode, flashlamp pumped, Nd:YAG, dye, argon, ytterbium, and Er:YAG among others. More than one laser or light source can be used in combination with RF to further enhance the effect. For example, a pulsed infra-red light source can be used to heat the skin surface, an Nd:YAG laser can be used to heat specific chromophores or dark matter below the surface of the skin, and RF current can be applied to a specific layer within or below the skin; the combination of which provides the optimal results for skin tightening, acne treatment, lipolysis, wart removal or any combination of these treatments.

Other energy modes besides or in addition to the optical energy described above can also be used in conjunction with RF current for these treatments. Ultrasound energy can be delivered either through the RF probes, through a face plate on the surface of the skin, or through a separate device. The ultrasound energy can be used to thermally treat the targeted tissue and/or it can be used to sense the temperature of the tissue being heated. A larger pulse of pressure can also be applied to the surface of the skin in addition to RF current to disrupt adipose tissue. Fat cells are larger and their membranes are not as strong as those of other tissue types so such a pulse can be generated to selectively destroy fat cells. In some cases, the multiple focused pressure pulses or shock waves can be directed at the target tissue to disrupt the cell membranes. Each individual pulse can have from 0.1 to 2.5 Joules of energy.

The energy supply unit 90 may also include an input/output (I/O) device that allows the physician to input control and processing variables, to enable the controller to generate appropriate command signals. The I/O device can also receive real time processing feedback information from one or more sensors associated with the device, for processing by the controller, e.g., to govern the application of energy and the delivery of processing fluid. The I/O device may also include a display, to graphically present processing information to the physician for viewing or analysis.

In some variations, the system 200 may also include an auxiliary unit 92 (where the auxiliary unit may be a vacuum source, fluid source, ultrasound generator, medication source, etc.) Although the auxiliary unit is shown to be connected to the energy supply, variations of the system 200 may include one or more auxiliary units 92 where each unit may be coupled to the power supply 90 and/or the treatment unit 202.

FIG. 2B illustrates a partial view of a working end of a treatment unit 202 where the treatment unit 202 engages against tissue 10 and the array 108 extends from a cartridge 100 into the tissue 10. The cooling device 234 also engages tissue 10 so that a cooling surface 216 cools tissue directly above the area of treatment. The illustrated figure also demonstrates another feature of the system where the cartridge 100 includes a tissue engaging surface 106 having a plane that forms an angle A with a plane of the array of probes 108. As described below, this configuration permits a larger treatment area as well as direct cooling of the tissue surface. The devices of the present invention may have an angle A of 15 degrees. However, the angle can range from anywhere between perpendicular to parallel with respect to the tissue surface. The tissue engaging surface 106 can also include any number of features to ensure adequate contact with tissue.

Although not shown, the tissue engagement surface may contain apertures or other features to allow improved engagement against tissue given the application of a vacuum. By drawing tissue against the tissue engaging surface the medical practitioner may better gauge the depth of the treatment. For example, given the relatively small sectional regions of the epidermis, dermis, and subcutaneous tissue, if a device is placed over an uneven contour of tissue, one probe pair may be not be placed at the sufficient depth. Accordingly, application of energy in such a case may cause a burn on the epidermis. Therefore, drawing tissue to the tissue engaging surface of the device increases the likelihood of driving the probes to a uniform depth in the tissue.

In such an example, the tissue engagement surface 106 can include small projections, barbs, or even an elastic resin to increase friction against the surface of tissue. These projections or features can grip or provide friction relative to the tissue in proximity of the target tissue. This grip or friction holds the tissue in place while the probes are inserted at an angle relative to the grip of the projections. In another variation, the tissue engaging surface can include contact or proximity sensors to ensure that any numbers of points along the tissue engaging surface are touching the surface of the target site prior to probe deployment and/or energy delivery.

FIG. 2B also shows the treatment unit 202 having an extension actuator 240 and a retraction actuator 242 which extend and retract the array 108 in the cartridge. The handle also contains a power control switch 244 that can start and stop delivery of energy. Clearly, the location, size, and construction of such actuators can vary. In addition, all actuators can be replaced by a single actuator. In yet another variation, actuation of the device can occur using a footswitch that is coupled to the control system.

As discussed below, the cooling device 234 includes a cooling plate or cooling surface 216. Optionally, the cooling surface can have a disposable cover that prevents direct tissue contact between the actual cooling surface and the target tissue. The cover can be a disposable, sterilized component that is discarded after each treatment or after each patient.

FIG. 2C shows another variation of a treatment system 200 according the principles described herein. The treatment system 200 generally includes a treatment unit 202 having a hand-piece 210 (or other member/feature that allows for manipulation of the system to treat tissue 10). The treatment unit 202 shown includes a faceplate 112 having a plurality of probes 104 (generally formed in an array 108) that extend from openings in the faceplate 112. The devices may comprise probe arrays of only a single probe up to considerably larger arrays. As noted above, the size of the array is determined by the target region that is intended for treatment. Additionally, the treatment unit 202 may or may not include an actuator 128 for driving the electrode array 108 from the faceplate 112. Alternative variations of the system 200 include actuators driven by the control system 90 or an auxiliary unit 92.

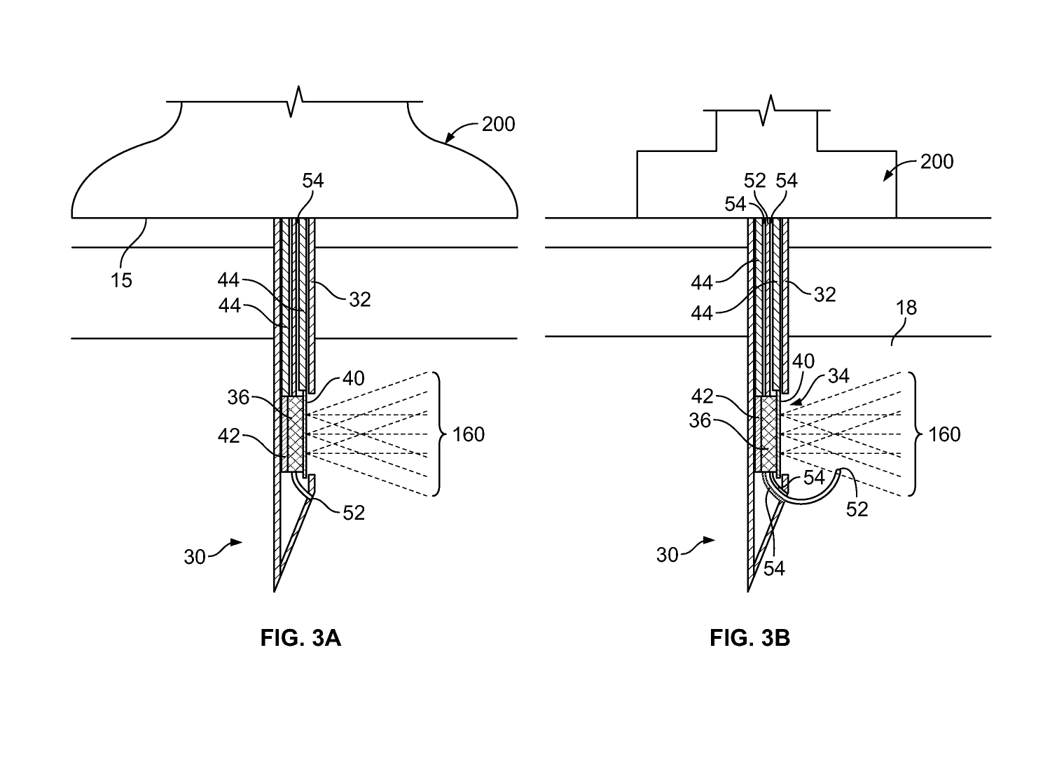

FIG. 3A shows a cross sectional view of a variation of a probe 30 of a treatment device 200 when inserted into tissue. The probe 30 can be any probe disclosed herein (including those entering the tissue at an oblique angle). A single probe is shown for illustrative purposes only. Clearly, any configuration of probes as disclosed herein can be used. In addition, although the following probes are shown entering tissue in a direction that is normal to the surface of the tissue, variations of the devices and methods disclosed herein contemplate oblique entry of the probes into tissue as discussed in further detail below.

As illustrated, the probes 30 shown have an active surface that provide therapeutic treatment in a targeted direction resulting in a zone of treatment that contains the greatest amount of energy delivered to the tissue. In the variation illustrated in FIGS. 3A and 3B, probe 30 includes an outer wall 32 which has an opening 34 on at least a portion of that wall 32. The opening 34 allows an energy delivery element 36 to apply energy from the probe to create a zone of treatment 160, such that the exposure of energy to tissue is non-uniform about the outer perimeter of the probe and greatest in the zone of treatment 160. As described below, any energy modality can be used to create the targeted zone of treatment.

As shown in FIG. 3A, the energy delivery element 36 comprises a piezoelectric crystal with a flexible transmitting cover membrane 40. The flexible membrane 40 can be coupled to at least one power delivery lead 44 and the other lead 44 is coupled to a conductive epoxy bed 42. The epoxy bed 42 secures the transducer 38 to one portion of the probe wall 34 and transmits power to the crystal 38. Power delivered to the crystal 38 from a power supply causes high frequency oscillation of the membrane 40 resulting in application of a high frequency acoustic energy into the surrounding tissue 10. This energy mechanically heats the dermal tissue to cause contraction and tightening of the collagen. As noted herein, this shrinking and tightening improves the appearance of the skin and reduces sagging and wrinkles.

FIG. 3A also illustrates an optional temperature sensor 52 and temperature sensing lead 54. Temperature sensor 52 can be any type of sensor such as a thermocouple, a thermistor, a ferrite bead, or a fluorescing dye. The temperature sensing lead 54 can be part of the sensor 52 or it can be a power supply line/wire from a power control module that transmits a signal to and from the sensor 52. In the case of a fluorescing dye, the sensor and lead may comprise a fiber optic line that provides illumination to the dye and transmit the reflected fluorescence back to a power control module. The use of the temperature sensor 52 and probe 30 of the current variation provide great advantages over other high frequency and ultra high frequency acoustic energy systems which direct the energy into the skin from the surface.

The use of the percutaneous probe 30 produces a desirable therapeutic effect with energy levels that are much lower than systems that are required to heat directly on the dermis rather than through the tough and rigid stratum corneum 15 and the sensitive epidermis. Furthermore, in some variations there is no need to sequentially or simultaneously cool the surface of the tissue to prevent the epidermis from heating too much as the energy is applied only to the dermis. In addition, the use of a temperature sensor 52 allows for a measurement of the adjacent dermal tissue in or near the treatment zone 160. This measurement provides a control mechanism for the power control module to adjust power delivery to the energy delivery element 36 to achieve the desired temperature/effect.

FIG. 3B shows an alternate variation of a probe 30 where a temperature sensor 52 is advance-able out of the probe 30 away from the probe wall 32 and into the area of dermis that is being directly treated by the energy element 36. The sensor 52 can be advanced directly into the zone of treatment 160 adjacent tissue. This configuration provides even more accurate temperature data for control of delivered energy. In additional variations, the temperature sensor 52 location can vary anywhere along the length of the probe 30 or even on the face of the treatment system 200. In addition, any number of temperature sensors 52 can be placed along or advanced from the probe/treatment system.

As noted above, the energy transfer element 36 delivers energy through an opening in a wall of the probe 30. In some variations, the opening can be covered with a material that allows energy to exit the probe but prevents tissue or other materials from entering the probe. Furthermore, the energy transfer element 36 can employ different modalities other than high frequency acoustic energy. For example, the energy transfer element 36 can comprise an illumination source, a microwave energy supply, a resistive heat source, an RF energy probe, or a cooling source. For example, the element can comprise a mono-polar or bi-polar RF energy electrode in such case the zone of treatment would comprise the path of electrical current flow through the probe. In another variation, the probe can be configured with insulation or reflectors to direct the energy from an otherwise multi-directional source (microwave, resistive heat source, cooling source, illumination) to create a zone of treatment.

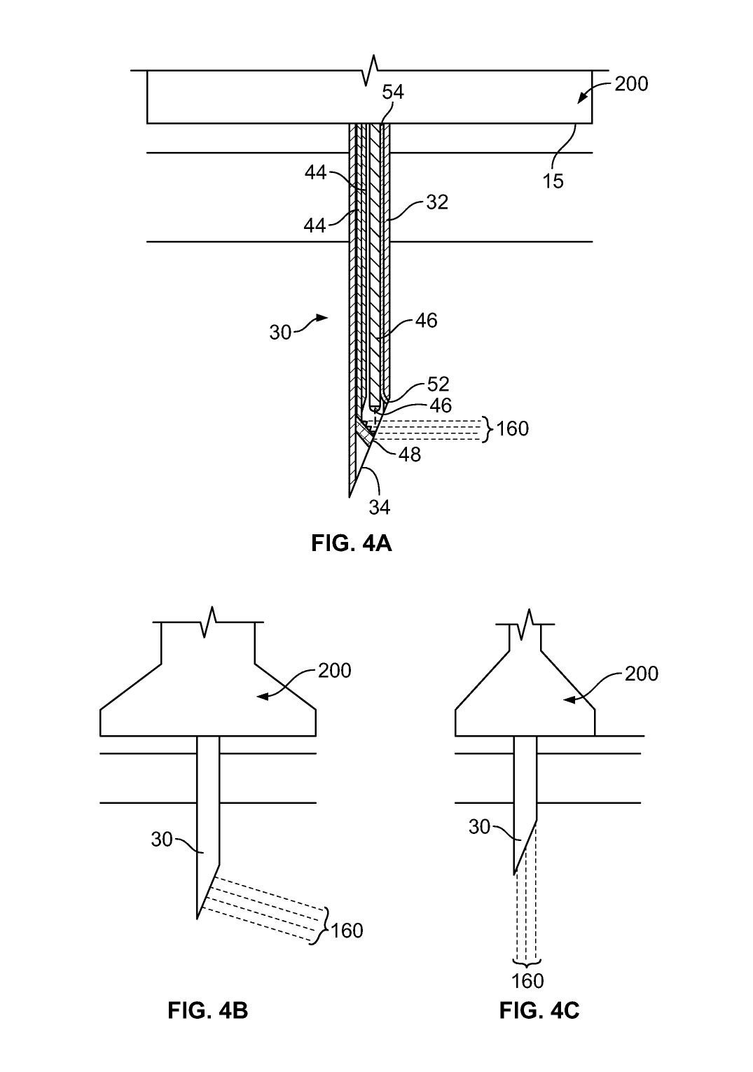

FIG. 4A illustrates one such variation of a probe 30 of a treatment system 200 employing an illumination energy transfer source. The illumination source can include a laser source or other light energy source that directs energy through the probe to the targeted tissue. As shown, the probe 30 contains an illumination source (e.g. a fiber optic) and includes a lens assembly 48 (or other deflection means) adjacent to an opening 34 in the probe 30. In this variation, the opening 34 is at a beveled distal tip. However, the opening can also be in a side-wall of the probe. The lens assembly 48 can be a digital micromirror device (DMD). The DMD can adjustably direct the light or laser energy out of the probe 30 to a zone of treatment 160 and into the target tissue. Variations of the system 200 can also include a temperature sensor 52 and electrical leads 44 to power and control the lens assembly 48. The lens assembly 48 can articulate to direct the energy into the tissue in any number of different angular directions as shown in FIGS. 4B and 4C.

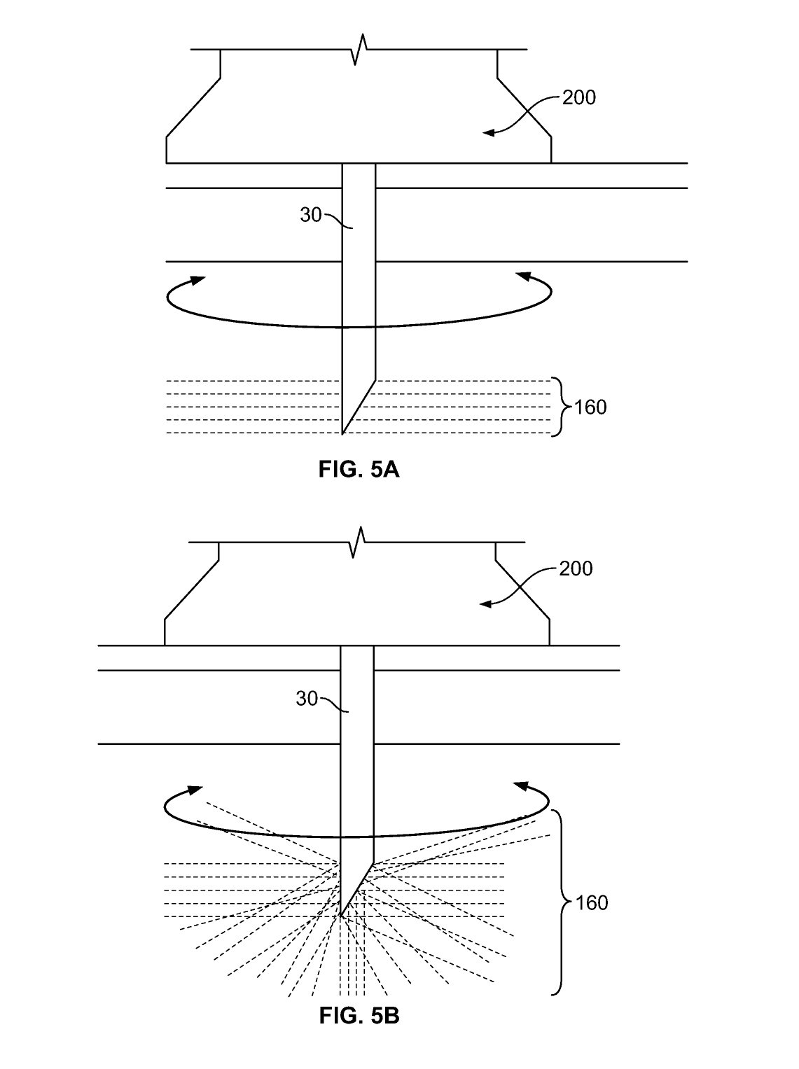

Furthermore, as shown in FIGS. 5A and 5B the probe or the energy delivery element can be rotated such that a greater portion of tissue can be targeted by the probe 30. In doing so, the zone of treatment 160 can selectively treat regions around the perimeter of the probe 30. In an additional variation, and as shown in FIG. 5B, the probe 30 can be rotated and the energy transfer element 36 can be articulated to create a larger zone of treatment 160 or to selectively treat regions around the probe 30.

In another variation of the device, an illumination source can be used to generate thermal energy that is applied to tissue rather than irradiate the tissue. For example, the mirror of the previous variations can be replaced with an optical absorbing emitter that is mounted on the probe. This emitter is configured to heat as is absorbs the light or laser energy. The emitter then conducts the heat to the target tissue via thermal conduction.

In additional variations the use of radio frequency, ultrasound, or microwave energy supplies can be directed towards an appropriate absorbing emitter that converts the delivered energy into thermal energy for treating the target tissue. Furthermore, the absorbing emitter can be composed of an inductive material which converts magnetic field energy into heat. This embodiment allows a smaller diameter delivery probe since the magnetic field can be produced outside of the target tissue and probe 30. In such a variation, there is no need to direct wires, antenna, fiber optics, transducers or other energy delivery methods through the inside of the probe 30 in order to apply the therapeutic treatment.

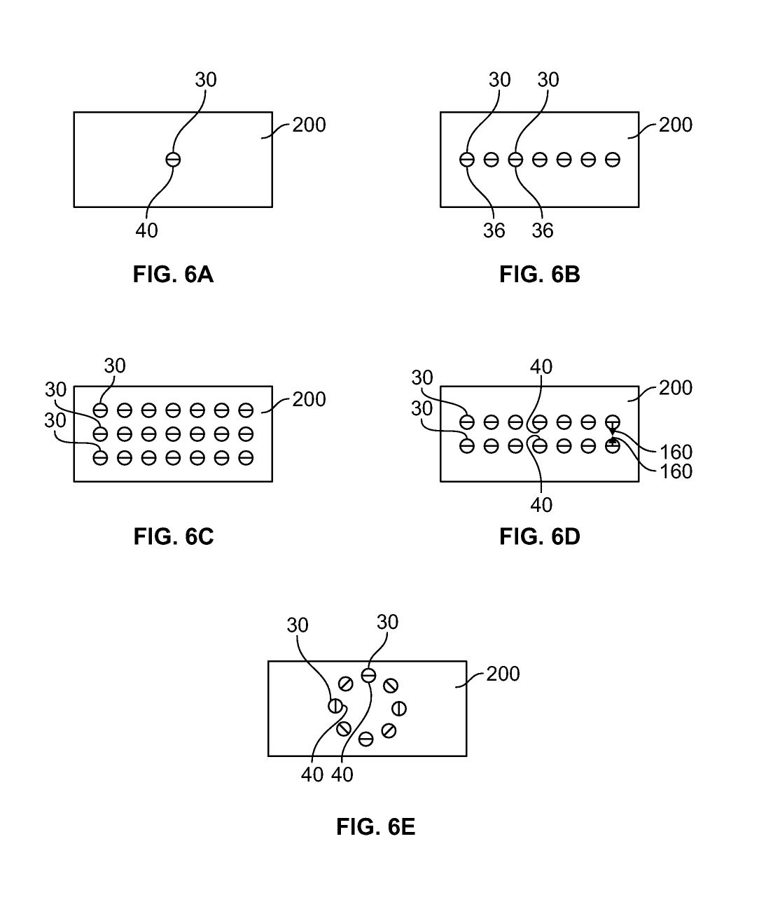

FIGS. 6A to 6E depict various probe 30 configurations for use in variations of the device. As shown in FIG. 6A, one variation of the system includes a single probe 30. However, a single row array, as shown in FIG. 6B or a multiple row array, as shown in FIG. 6C are also within the scope of the disclosure. As discussed below, the probes may be staggered such that the treatment zones affect varying depths of tissue as well.

FIGS. 6D and 6E illustrate another variation of the system 200 where openings 34 with membranes 40 on adjacent probes 30 face one another so that the zone of treatment 160 from adjacent probes 30 intersects to treat tissue. One such benefit of this configuration is that the power generated by each probe alone can be reduced such that a region of tissue is only treated in the intersecting zone between adjacent probes. For example, the power from one probe 30 can be set sufficiently low to insufficiently heat the tissue to a therapeutic level. However, in the region of treatment created by intersecting treatment zones, the generated heat is sufficient to create the desired effect.

In addition, FIG. 6E shows a circular array of probes 30 having openings 34 with membranes 40 or energy directors that focus on the center of the array as shown in FIG. 6E. Again this configuration allows for the delivery of even lower levels of energy form any one probe 30. Accordingly, the device will only treat tissue when all of the probes are energized simultaneously so that the combined focused energy is sufficient to create a therapeutic effect. These array variations allow for even more precise energy delivery than is possible with surface delivered devices.

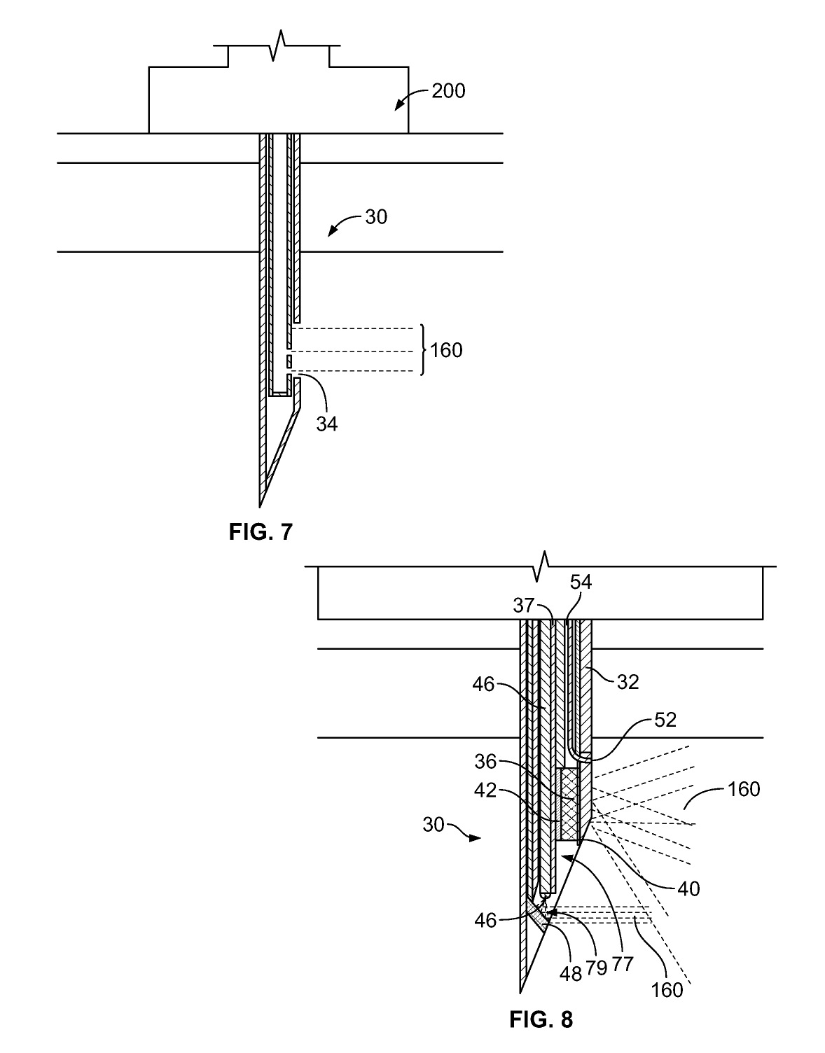

FIG. 7 shows yet another alternative variation for delivering energy to the targeted tissue. In this variation the probe 30 includes openings 34 that permit delivery of a fluid. Clearly, the probe can include one or more additional openings located anywhere along the probe. The probe 30 can be configured to produce a jet of fluid when pressurized. This jet or jets of fluid create a treatment zone 160 to produce a therapeutic effect in tissue. Any fluid, such as sterile saline, when delivered at a sufficient velocity and pressure can mechanically disrupt the collagen of the dermal layer creating a therapeutic effect. Although the probe 30 can directly deliver the fluid, other configurations are possible. For example, the probe can include a fluid delivery member 58 located within a body of the probe 30.

FIG. 8 shows another alternative variation of a probe for use with devices and methods disclosed herein. The illustrated probe 30 two lumens 77 and 79. The first lumen 77 includes a source of ultrasound energy. Specifically the probe is composed of an outer wall 32 which has an opening 34 on at least a portion of the wall 32. As described above, the probe can include a piezo electric crystal 38 with a flexible transmitting cover membrane 40. The flexible membrane is coupled to one of the power delivery leads 44 and the other lead 44 is coupled to a conductive epoxy bed 42. The epoxy bed 42 secures the crystal 38 to an interior of the probe and transmits power to the crystal 38. Delivery of power to the crystal 38 causes the flexible membrane 40 to oscillate direct acoustic energy into the target tissue. The variation also can include a temperature sensor 52 and temperature sensing lead 54 for monitoring target tissue temperature and controlling energy delivery.

The second lumen 79 of the probe can include a second type of energy delivery device. In this variation, the second lumen 79 includes elements for delivering laser or light energy to the targeted tissue. The lumen 79 contains a fiber optic 46 which has a lens assembly 48 at the distal tip. Distal of the lens assembly 48 can be a digital micromirror device (DMD). The lens assembly 48 can direct the light or laser energy out of the cannula opening 34 and into the target tissue as discussed above.

The combination of the two energy modalities, laser and ultrasound, directed to the target tissue can provided an enhanced therapeutic effect to the target tissue. Clearly any number of energy modalities can be combined within a single probe 30. Furthermore, the probe can include two separate zones of treatment given each energy modality.

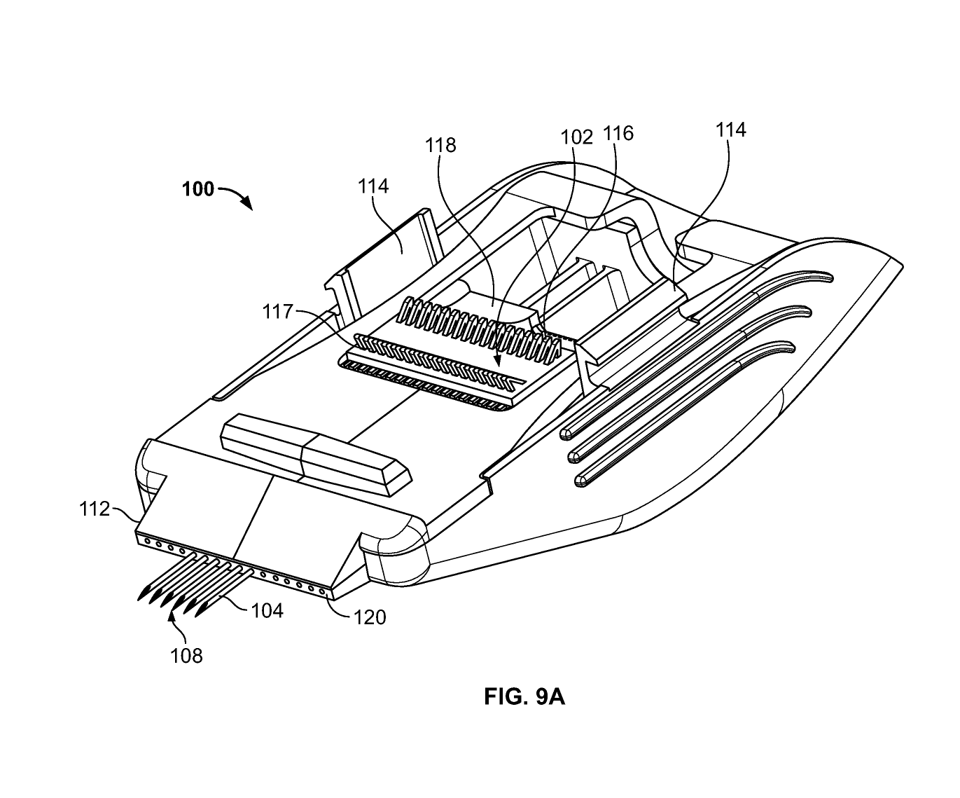

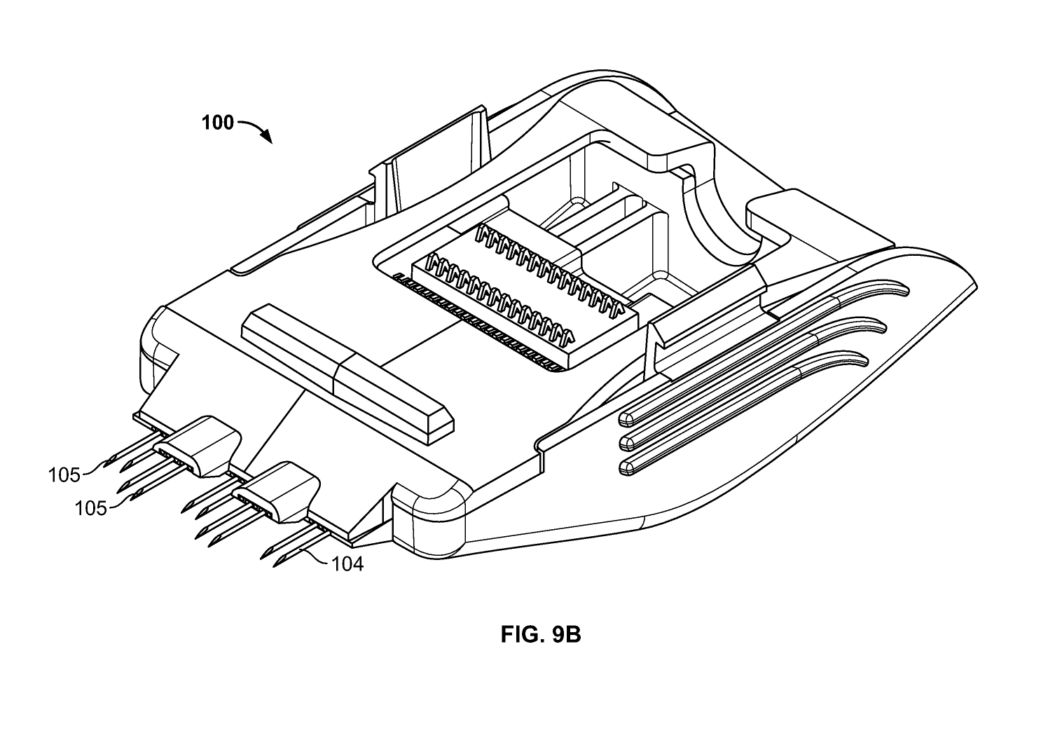

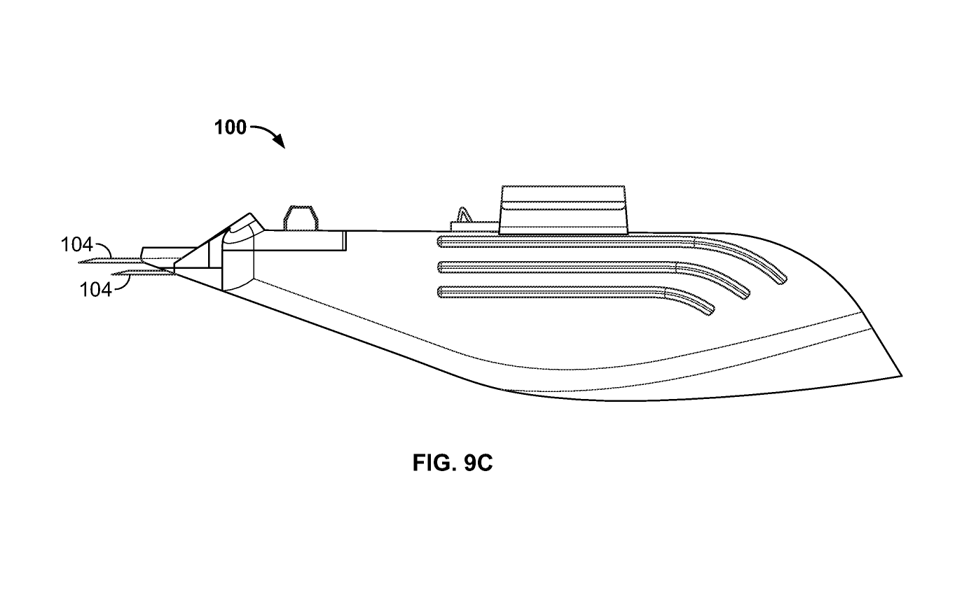

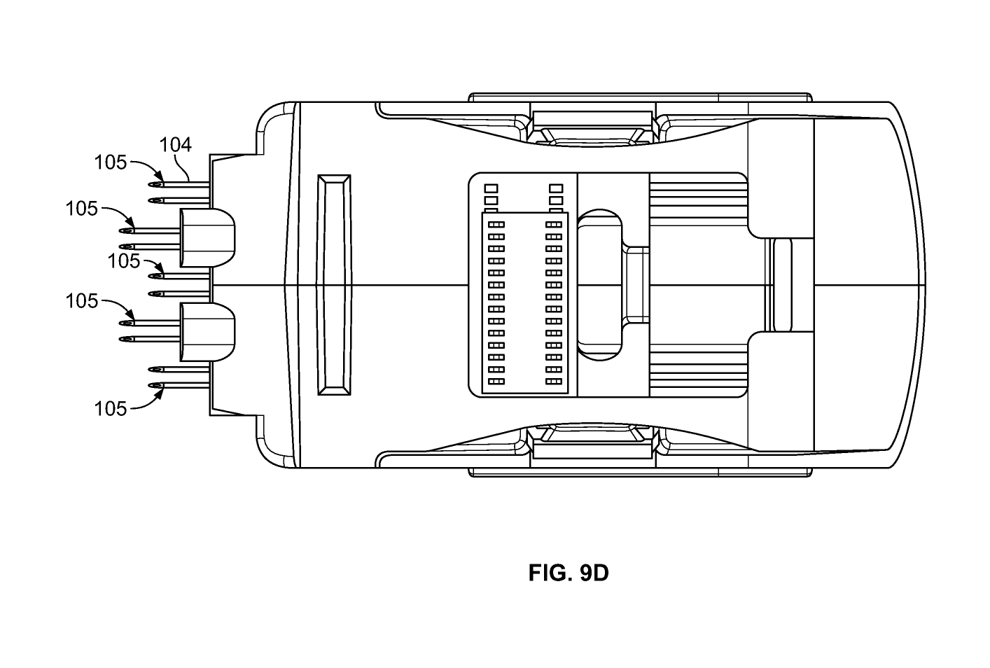

FIG. 9A illustrates one variation of a removable cartridge body 100 for use with the present system. As shown, the cartridge body 100 includes retention fasteners 114 allowing for coupling with the device body as well as removal from the device body. Again, any number of structures can be incorporated into the device to permit removable coupling of the cartridge body 100 to a treatment unit. The probes described above can be combined into the various cartridge bodies 100 shown herein.

The cartridge body 100 further includes a probe assembly 102 that is moveable or slidable within the cartridge body 100. The mode of movement of the actuator can include those modes that are used in such similar applications. Examples of these modes include, sliding, rotation, incremental indexing (via a ratchet-type system), stepping (via an step-motor) Accordingly, the probe assembly 102 can include a coupling portion or structure 118 that mates with an actuating member in the device body. In the illustrated example, the probe assembly 102 is in a treatment position (e.g., the array 108 extends from the cartridge 100 allowing for treatment). The probe assembly 102 includes any number of probes 104 that form an array 108 and are extendable and retractable from a portion 104 of the cartridge 100 (as noted above, the probes can alternatively extend from the device body, or other parts of the system). As noted above, although the illustrated example shows an array 108 of 1×6 probes 104, the array can comprise any dimension of M×N probes where the limits are driven by the nature of the treatment site as well as the type of energy delivery required.

FIG. 9A also shows the probes 104 in the probe assembly 102 as having connection or contact portions 116 that couple to a connection board on a treatment unit to provide an electrical pathway from the power supply to the probes 104. In the illustrated variation, the probe assembly 102 as well as the connection portions 116 moves. Such a feature allows for selective connection of the probes with the power supply. For example, in certain variations of the system, the probes are only coupled to the power supply when in a treatment position and are incapable of delivering energy when in a retracted position. In another variation, the probe assembly and connection board are configured to permit temperature detection at all times but only energy delivery in the treatment position. Such customization can prevent energy delivery in an unintended location, for example, when the probes have an insulation that only allows energy delivery at the distal tip and the intended location of energy delivery is at specific depth in the target tissue that corresponds to the length of the extended probe the probe cannot delivery energy to an unintended shallower location when it is not fully extended. However, any number of variations is possible. For example, the system can be configured so that the probes can be energized whether in the treatment or retracted positions.

The connection portions 116 can be fabricated in any number of configurations as well. For example, as shown, the connection portions 116 comprise spring contacts or spring pins of the type shown. Accordingly, the connection portions 116 can maintain contact with a corresponding contact point trace on a connection board during movement of the probe assembly 102

FIG. 9A shows the front portion 112 of the cartridge 100 as having multiple guiding channels 120. These channels 120 can support and guide the probes 104 as they advance and retract relative to the cartridge 100. The channels 120 can also be configured to provide alternate energy treatments to the surface of the tissue as well as suction or other fluids as may be required by a procedure. One benefit is that a single cartridge design can be configured to support a variety of probe array configurations. For example rather than the array of six (6) probes as shown, the channels 120 can support any number of probes (the illustrated example shows a maximum of sixteen (16) but such a number is for exemplary purposes only). Furthermore, the channels 120 need not be only in a linear arrangement as shown, but could be in 1, 2, 3 or more rows or in a random configuration.

FIG. 9B shows a perspective view of another variation of a probe assembly. In this variation, the probes 104 are staggered or offset such that adjacent probe pairs 105 do not form a linear pattern. One such benefit of this configuration is to overcome the creation of a “line effect” in tissue. For example, an array of probes arranged in a single line can possibly result in a visible line in tissue defined by the entry points of adjacent and parallel probes. In the variation of FIG. 3C, staggering or offsetting the probes prevents the “line effect” from occurring.

FIG. 9C shows a side view of the variation of FIG. 9B. As shown, the probes 104 are offset to minimize the chance of forming a single continuous line in tissue by penetration of a set of linearly arranged probes. Clearly, other configuration can also address the “line effect”. For example, the spacing between adjacent probes can be increased to minimize a “line effecf” but to still permit efficacy of treatment. In addition, although the illustrated example shows two lines of probes, variations of the device include probes 104 that form more than two rows of probes.

FIG. 9D shows a top view of the cartridge variation of FIG. 3C. The variation illustrated shows that the plurality of probes comprises a plurality of probe pairs 105. As noted above, the probe pairs 105 can be vertically offset from an adjacent probe pair (as shown in FIG. 9C) so that insertion of probe pairs into the tissue does not create a continuous line of insertion points. Moreover, and as shown in FIG. 9D the probes 104 can be axially offset (such that an end of the probe) extends a greater distance than an end of an adjacent probe or probe pair. As noted herein, axially offsetting the probes allows for a uniform insertion depth when measured relative to a tissue engaging surface of the cartridge.

Commonly assigned U.S. patent application Ser. No. 12/025,924 filed on Feb. 1, 2008 entitled CARTRIDGE ELECTRODE DEVICE, the entirety of which is incorporated by reference herein, includes additional details of removable cartridge assemblies for use with the systems described herein.

The present systems may apply treatments based upon sensing tissue temperature conditions as a form of active process feedback control. Alternatively, those systems relying on conduction of energy through the tissue can monitor changes in impedance of the tissue being treated and ultimately stop the treatment when a desired value is obtained. In another variation, the delivery of energy can depend on whether impedance is within a certain range. Such impedance monitoring can occur during energy delivery and attenuate power if the dynamically measured impedance starts to exceed a given value or if the rate of increase is undesirably high. Yet another mode of energy delivery is to provide a total maximum energy over a duration of time.

As noted herein, temperature or other sensing may be measured beneath the epidermis in the dermis region. As shown above, each probe may include a sensor or a sensor can be placed on a probe-like structure that advances into the tissue but does not function as an energy delivery probe. In yet another variation, the sensors may be a vertically stacked array (i.e. along the length of the probe) of sensors to provide data along a depth or length of tissue.

Applying the therapeutic treatment in the dermal layer produces a healing response caused by thermally denaturing the collagen in the dermal layer of a target area. As noted herein, systems according to the present invention are able to provide a desirable effect in the target area though they use a relatively low amount of energy when compared to systems that treat through the epidermis. Accordingly, systems of the present invention can apply energy in various modes to improve the desired effect at the target area.

In one mode, the system can simply monitor the amount of energy being applied to the target site. This process involves applying energy and maintaining that energy at a certain pre-determined level. This treatment can be based on a total amount of energy applied and/or application of a specific amount of energy over a set period of time. In addition, the system can measure a temperature of the target site during the treatment cycle and hold that temperature for a pre-determined amount of time. However, in each of these situations, the system does not separate the time or amount of energy required to place the target site in the desired state from the time or amount of energy required to hold the target site in the desired state. As a result, the time or amount of energy used to place the target in a desired state (e.g., at a pre-determined temperature) is included in the total treatment cycle. In some applications, it may be desirable to separate the portion of the treatment cycle required to elevate the target to a pre-determined condition from the portion of the treatment cycle that maintains the target site at the pre-determined conditions.

For example, in one variation, the system can maintain a temperature of the target site at a pre-determined treatment temperature during a pre-determined cycle or dwell time. The system then delivers energy to maintain the target site at the treatment temperature. Once the target site reaches the treatment temperature, the system then maintains this condition for the cycle or dwell time. This variation allows for precise control in maintaining the target site at the pre-determined temperature. In another variation, the system can monitor the amount of power applied to the target site for a specific dwell time. By continuously measuring current and output voltage, the system can calculate both the impedance changes and the delivered power levels. With this method a specific amount of power can be delivered to the target tissue for a specified amount of time. In addition, the above variations can be combined with various methods to control time, temperature or energy parameters to place the tissue in the desired state. For example, the system can employ a specified ramp time or maximum energy to achieve the pre-determined treatment temperature. Such a variation can create a faster or slower ramp to the treatment temperature.

Although the treatment of tissue generally relies on energy to affect the tissue, the mere act of inserting the probe array into tissue can also yield therapeutic benefits. For instance, the mechanical damage caused by placement of the probes also produces an adjunct healing response. The healing response to injury in the skin tissue can contribute to the production of new collagen (collagenesis) that can further improve the tone or appearance of the skin. Accordingly, in one variation a medical practitioner may opt to use the methods and systems to create mechanical injury to tissue by placing probes into target areas without thermal treatment to induce a healing response in the targeted area. Accordingly, the invention is not limited to application of energy via the probes.

The low energy requirements of the system present an additional advantage since the components on the system undergo less stress than those systems needing higher amounts of energy. In those systems requiring higher energy, RF energy is often delivered in a pulsed fashion or for a specific duty cycle to prevent stressing the components of that system. In contrast, the reduced energy requirements of the present system allow for continual delivery of RF energy during a treatment cycle. In another variation, the duty cycle of variations of the present system can be pulsed so that temperature measurements can be taken between the pulsed deliveries of energy. Pulsing the energy delivery allows for an improved temperature measurement in the period between energy deliveries and provides precise control of energy delivery when the goal of the energy delivery is to reach a pre-determined temperature for a pre-determined time.

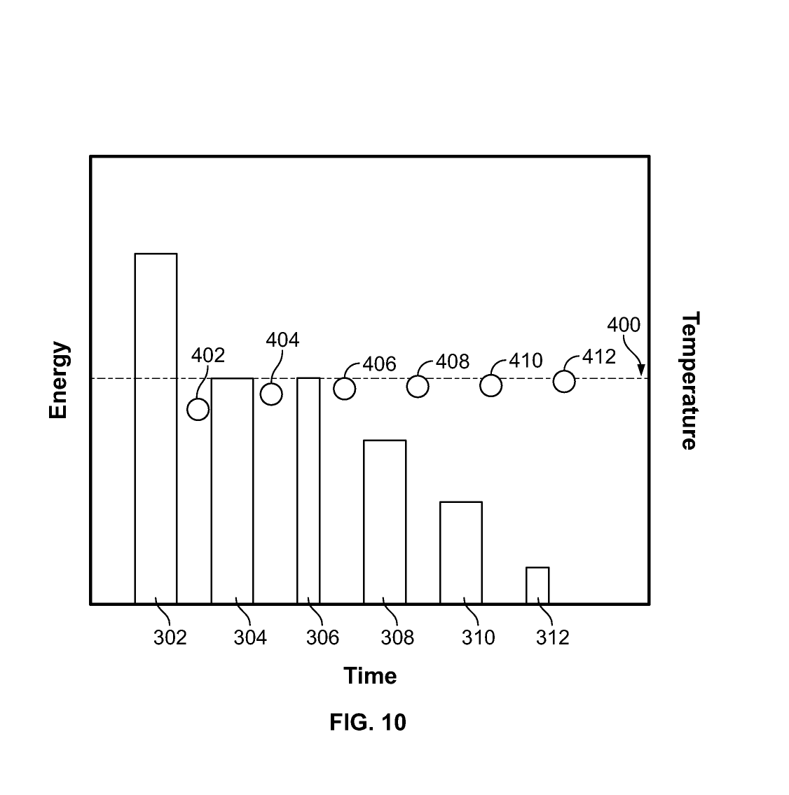

FIG. 10 illustrates a graph of energy delivery and temperature versus time. As shown, the pulses or cycles of energy are represented by the bars 302, 304, 306, 308, 310, 312. Each pulse has a parameter, including amount of energy, duration, maximum energy delivered, energy wave form or profile (square wave, sinusoidal, triangular, etc), current, voltage, amplitude, frequency, etc. As shown in the graph, measurements are taken between pulses of energy. Accordingly, between each pulse of energy delivery one or more temperature sensor(s) near the probe obtains a temperature measurement 402, 404, 406, 408, 410, 412. The controller compares the measured temperature to a desired temperature (illustrated by 400). Based on the difference, the energy parameters are adjusted for the subsequent energy pulse. Measuring temperature between pulses of energy allows for a temperature measurement that is generally more accurate than measuring during the energy delivery pulse. Moreover, measuring between pulses allows for minimizing the amount of energy applied to obtain the desired temperature at the target region.

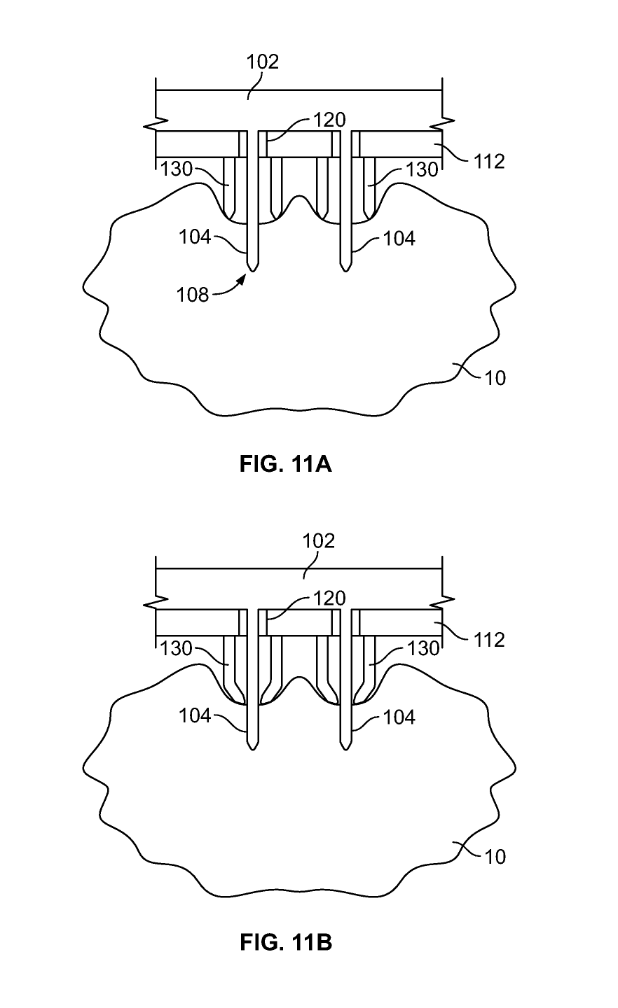

FIG. 11A illustrates an aspect for use with the variations of the devices described herein that eases insertion of probes into tissue. In this example, the probes 104 advance through an introducer member or cannula 130 located on the front face 112 of a cartridge. The cannula 130 places tissue 10 in a state of tension (also called “traction”). In this variation the introducer/cannula 130 is located about each channel 120 in the cartridge.

As shown, once the introducer member 130 engages tissue 10, the tissue first elastically deforms as shown. Eventually, the tissue can no longer deflect and is placed in traction by the introducer members 130. As a result, the probes 104 more readily penetrate the tissue.

FIG. 11B illustrates another variation of the introducer member 130 that is tapered inwards toward the probes so that the opening at the distal end closely fits around the probe.

In another variation, insertion of the array 108 can consist of 2 or more steps. In the first step the actuation of the extension presses the channels 120 against the target tissue to create a state of traction. Further actuation advances the array 108 through the channels 120 and into the target tissue. Since the target tissue is under traction, the array requires less force to penetrate the tissue. In another variation, the channels 120 can be individual cannulas that extend from the distal face of the cartridge. Such a configuration produces traction on a smaller portion of target tissue. Alternatively, the two step extension process can be composed of a first step which extends small projections out of the tissue engaging surface of the cartridge in a direction that is substantially opposite of the direction of probe extension which occurs in the second step. This alternative creates more traction which further eases insertion of the probes as the target tissue is stretched in opposite directions.

In those variations of the device using an RF energy modality, the probes 104 can be arranged in a pair configuration. In a bi-polar configuration one probe serves a first pole, while the second probe serves as the second pole (it is also common to refer to such probes as the active and return probes). The spacing of probe pairs is sufficient so that the air of probes is able to establish a treatment current path therebetween for the treatment of tissue. However, adjacent probe pairs can be spaced sufficiently to minimize the tendency of current flowing between the adjacent pairs. Typically, each probe pair is coupled to a separate power supply or to a single power supply having multiple channels for each probe pair.

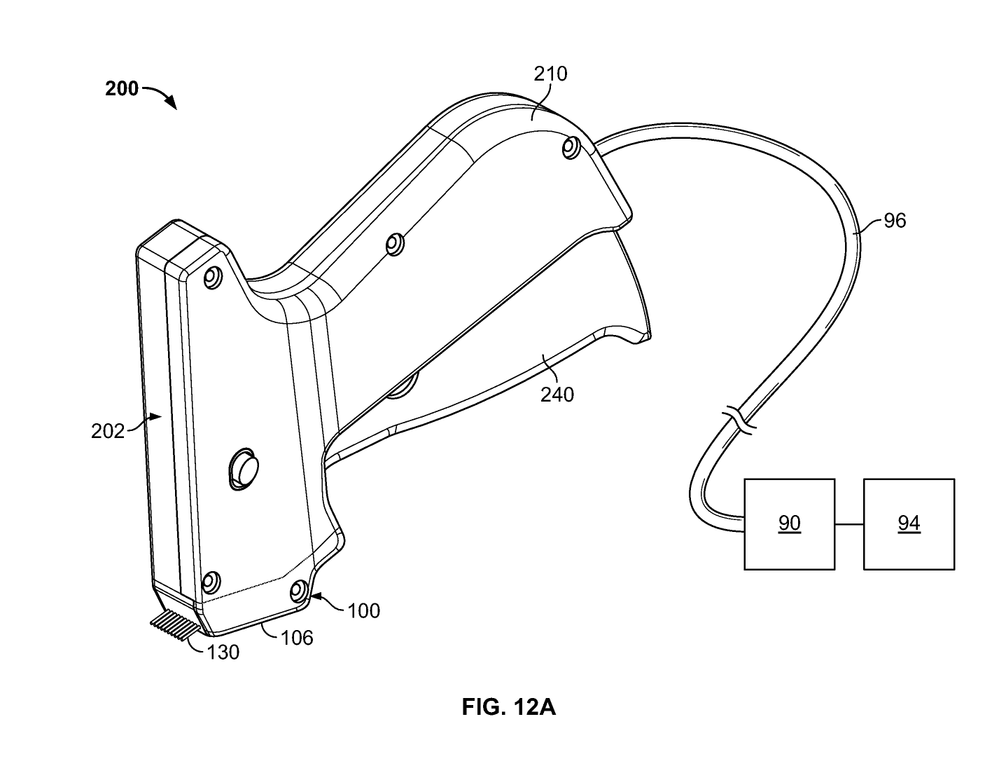

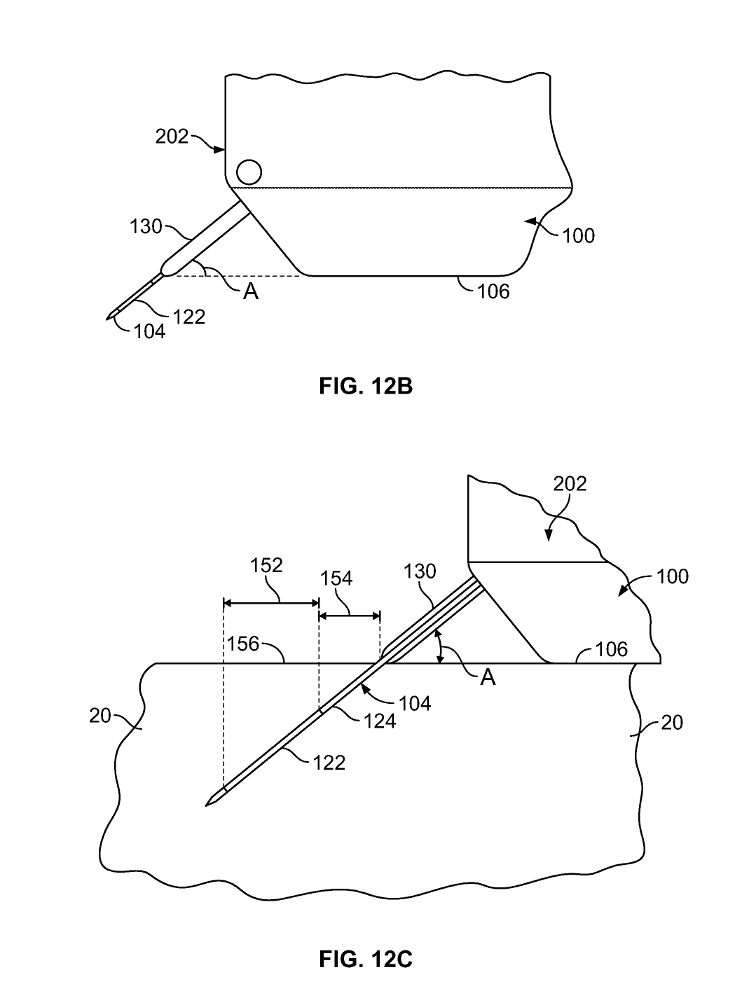

FIG. 12A illustrates another variation of a system 200 for use in accordance with the principles discussed herein. In this variation, the system 200 includes a treatment unit 202 having a cartridge 100 from which a probe or introducer member 130 extends at an oblique angle relative to a tissue engagement surface 106. As described below, the ability to insert the probes (not shown) into the tissue at an oblique angle increases the treatment area and allows for improved cooling at the tissue surface. Although the variation only shows a single array of introducers for probes, variations of the invention may include multiple arrays of probes. In addition, the devices and systems described below may be combined with the features described herein to allow for improved penetration of tissue. The devices of the present invention may have an angle A of 15 degrees. However, the angle may be anywhere from ranging between 5 and 85 degrees.

Although the introducer member 130 is shown as being stationary, variations of the device include introducer members that are slidable on the probes. For example, to ease insertion of the probe, the probe may be advanced into the tissue. After the probe is in the tissue, the introducer member slides over the probe to a desired location. Typically, the introducer member is insulated and effectively determines the active region of the probe. In another variation using RF energy, the introducer member may have a return probe on its tip. Accordingly, after it advances into the tissue, application of energy creates current path between the probe and the return probe on the introducer.

The treatment unit 202 of the device 200 may also include a handle portion 210 that allows the user to manipulate the device 200. In this variation, the handle portion 210 includes a lever or lever means 240 that actuates the probes into the tissue (as discussed in further detail below).

As discussed above, the device 200 can be coupled to a power supply 90 with or without an auxiliary unit 94 via a connector or coupling member 96. In some variations of the device, a display or user interface can be located on the body of the device 200 as discussed below.

FIG. 12B illustrates a partial side view of the probes 104 and tissue engaging surface 106 of the probe device of FIG. 12A. As shown, the probes 104 extend from the cartridge 100 through the introducer 130. In alternate variations, the probes can extend directly from the body of the device or through extensions on the device.

As shown, the probes 104 are advanceable from the cartridge (in this case through the introducers 130) at an oblique angle A as measured relative to the tissue engagement surface 106. The tissue engagement surface 106 allows a user to place the device on the surface of tissue and advance the probes 104 to the desired depth of tissue. Because the tissue engagement surface 106 provides a consistent starting point for the probes, as the probes 104 advance from the device 202 they are driven to a uniform depth in the tissue.

For instance, without a tissue engagement surface, the probe 104 may be advanced too far or may not be advanced far enough such that they would partially extend out of the skin. As discussed above, either case presents undesirable outcomes when attempting to treat the dermis layer for cosmetic effects. In cases where the device is used for tumor ablation, inaccurate placement may result in insufficient treatment of the target area.

FIG. 12C illustrates a magnified view of the probe entering tissue 20 at an oblique angle A with the tissue engaging surface 106 resting on the surface of the tissue 20. As is shown, the probe 104 can include an active area 122. Generally, the term “active area” refers to the part of the probe through which energy is transferred to or from the tissue. For example, the active area could be a conductive portion of an probe, it can be a resistively heated portion of the probe, or even comprise a window through which energy transmits to the tissue. Although this variation shows the active area 122 as extending over a portion of the probe, variations of the device include probes 104 having larger or smaller active areas 122.

In any case, because the probes 104 enter the tissue at an angle A, the resulting region of treatment 152, corresponding to the active area 122 of the probe is larger than if the needle were driven perpendicular to the tissue surface. This configuration permits a larger treatment area with fewer probes 104. In addition, the margin for error of locating the active region 122 in the desired tissue region is greater since the length of the desired tissue region is greater at angle A than if the probe were deployed perpendicularly to the tissue.

As noted herein, the probes 104 may be inserted into the tissue in either a single motion where penetration of the tissue and advancement into the tissue are part of the same movement or act. However, variations include the use of a spring mechanism or impact mechanism to drive the probes 104 into the tissue. Driving the probes 104 with such a spring-force increases the momentum of the probes as they approach tissue and facilitates improved penetration into the tissue. As shown below, variations of the devices discussed herein may be fabricated to provide for a dual action to insert the probes. For example, the first action may comprise use of a spring or impact mechanism to initially drive the probes to simply penetrate the tissue. Use of the spring force or impact mechanism to drive the probes may overcome the initial resistance in puncturing the tissue. The next action would then be an advancement of the probes so that they reach their intended target site. The impact mechanism may be spring driven, fluid driven or via other means known by those skilled in the art. One possible configuration is to use an impact or spring mechanism to fully drive the probes to their intended depth.

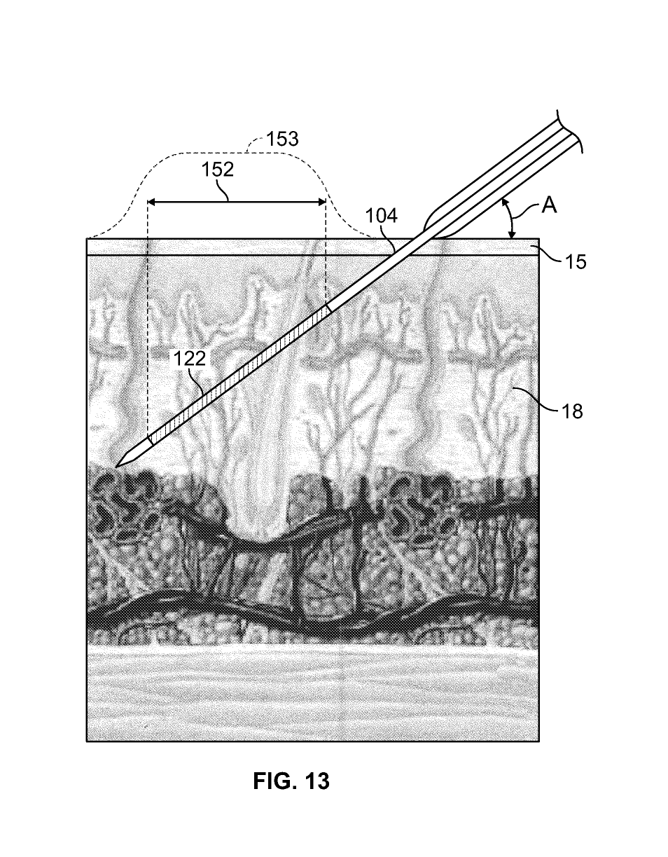

FIG. 13 illustrates an example of the benefit of oblique entry when the device is used to treat the dermis 18. As shown, the length of the dermis 18 along the active region 122 is greater than a depth of the dermis 18. Accordingly, when trying to insert the probe in a perpendicular manner, the shorter depth provides less of a margin for error when trying to selectively treat the dermis region 18. As discussed herein, although the figure illustrates treatment of the dermis to tighten skin or reduce wrinkles, the device and methods may be used to affect skin anomalies 153 such as acne, warts, sebaceous glands, tattoos, or other structures or blemishes. In addition, the probe may be inserted to apply energy to a tumor, a hair follicle, a fat layer, adipose tissue, SMAS, a nerve or a pain fiber or a blood vessel. As noted herein, the probes shown can include any variation of probe disclosed above.

Inserting the probe at angle A also allows for direct cooling of the surface tissue. As shown in FIG. 12C, the area of tissue on the surface 156 that is directly adjacent or above the treated region 152 (i.e., the region treated by the active area 122 of the probe 104) is spaced from the entry point by a distance or gap 154. This gap 154 allows for direct cooling of the entire surface 156 adjacent to the treated region 152 without interference by the probe or the probe mounting structure. In contrast, if the probe were driven perpendicularly to the tissue surface, then cooling must occur at or around the perpendicular entry point.

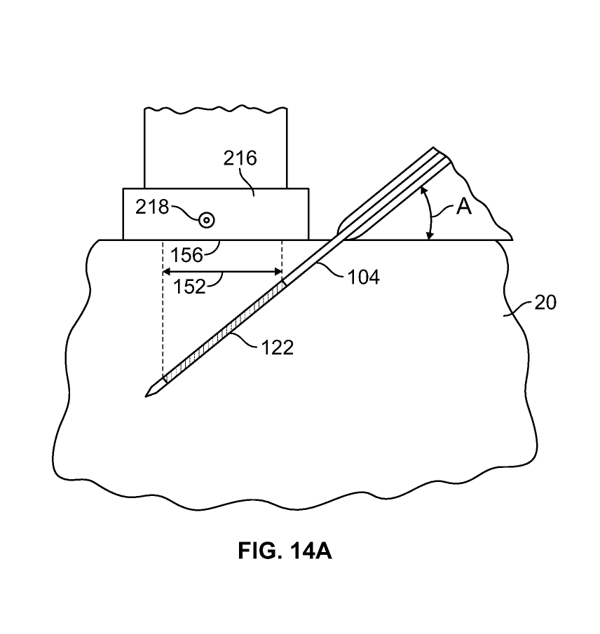

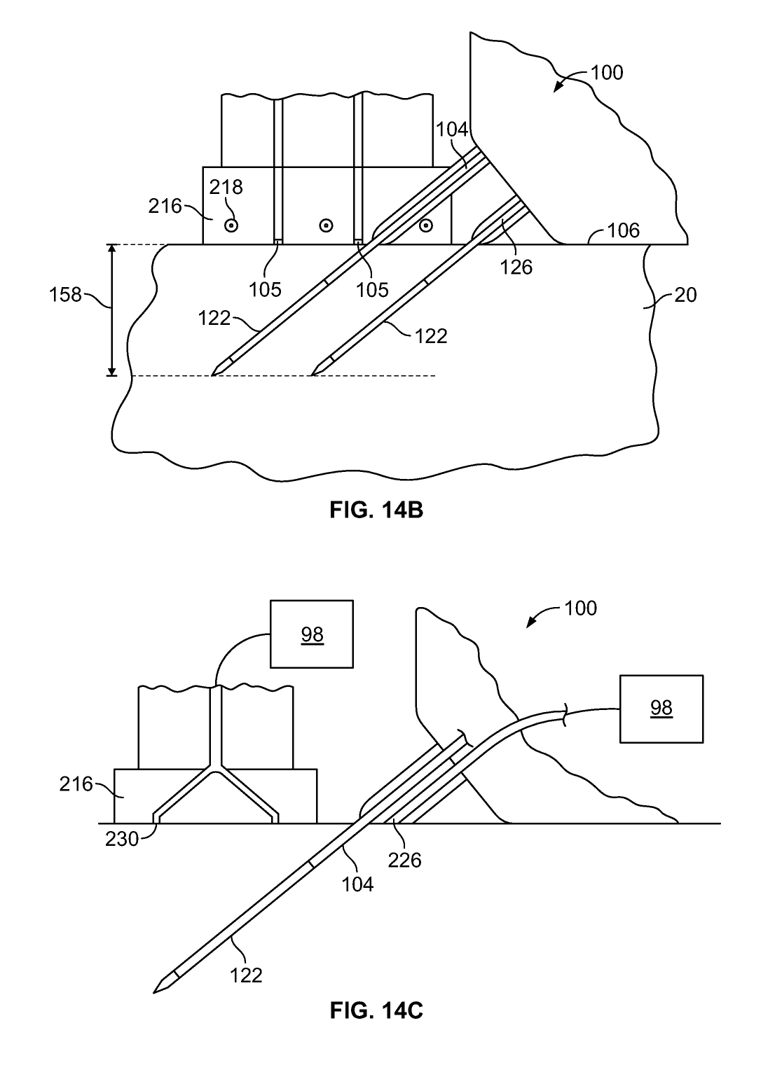

FIG. 14A illustrates one example of a cooling surface 216 placed on body structure or tissue 20. As shown, the probe 104 enters at an oblique angle A such that the active region 122 of the probe 104 is directly adjacent or below the cooling surface 216. In certain variations, the cooling surface 216 may extend to the entry point (or beyond) of the probe 104. However, it is desirable to have the cooling surface 216 over the probe's active region 122 because the heat generated by the active region 122 will have its greatest effect on the surface at the surface location 156. In some variations, devices and methods described herein may also incorporate a cooling source in the tissue engagement surface.

The cooling surface 216 and cooling device may be any cooling mechanism known by those skilled in the art. For example, it may be a manifold type block having liquid or gas flowing through for convective cooling. Alternatively, the cooling surface 216 may be cooled by a thermoelectric cooling device (such as a fan or a Peltier-type cooling device). In such a case, the cooling may be driven by energy from the probe device thus eliminating the need for additional fluid supplies. One variation of a device includes a cooling surface 216 having a temperature detector 218 (thermocouple, RTD, optical measurement, or other such temperature measurement device) placed within the cooling surface. The device may have one or more temperature detectors 218 placed anywhere throughout the cooling surface 216 or even at the surface that contacts the tissue.

In one application, the cooling surface 216 is maintained at or near body temperature. Accordingly, as the energy transfer occurs causing the temperature of the surface 156 to increase, contact between the cooling surface 216 and the tissue 20 shall cause the cooling surface to increase in temperature as the interface reaches a temperature equilibrium. Accordingly, as the device's control system senses an increase in temperature of the cooling surface 216 additional cooling can be applied thereto via increased fluid flow or increased energy supplied to a Peltier-type device. The cooling surface can also pre-cool the skin and underlying epidermis prior to delivering the therapeutic treatment. Alternatively, or in combination, the cooling surface can cool the surface and underlying epidermis during and/or subsequent to the energy delivery where such cooling is intended to maintain the epidermis at a specific temperature below that of the treatment temperature. For example the epidermis can be kept at 30 degrees C. when the target tissue is raised to 65 degrees C.

When treating the skin, it is believed that the dermis should be heated to a predetermined temperature condition, at or about 65 degree C., without increasing the temperature of the epidermis beyond 42 degree C. Since the active area of the probe designed to remain beneath the epidermis, the present system applies energy to the dermis in a targeted, selective fashion, to dissociate and contract collagen tissue. By attempting to limit energy delivery to the dermis, the configuration of the present system also minimizes damage to the epidermis.

While the cooling surface may comprise any commonly known thermally conductive material, metal, or compound (e.g., copper, steel, aluminum, etc.). Variations of the devices described herein may incorporate a translucent or even transparent cooling surface. In such cases, the cooling device will be situated so that it does not obscure a view of the surface tissue above the region of treatment.

In one variation, the cooling surface can include a single crystal aluminum oxide (Al₂O₃). The benefit of the single crystal aluminum oxide is a high thermal conductivity optical clarity, ability to withstand a large temperature range, and the ability to fabricate the single crystal aluminum oxide into various shapes. A number of other optically transparent or translucent substances could be used as well (e.g., diamond, other crystals or glass).

FIG. 14B illustrates another aspect for use with variations of the devices and methods described herein. In this variation, the cartridge 100 includes two arrays of probes 104, 126. As shown, the first plurality 104 is spaced evenly apart from and parallel to the second plurality 126 of probes. In addition, as shown, the first set of probes 104 has a first length while the second set of probes 126 has a second length, where the length of each probe is chosen such that the sets of probes 104, 126 extend into the tissue 20 by the same vertical distance or length 158. Although only two arrays of probes are shown, variations of the invention include any number of arrays as required by the particular application. In some variations, the lengths of the probes 104, 126 are the same. However, the probes will be inserted or advanced by different amounts so that their active regions penetrate a uniform amount into the tissue. As shown, the cooling surface may include more than one temperature detecting element 218.

FIG. 14B also illustrates a cooling surface 216 located above the active regions 122 of the probes. In such a variation, it may be necessary for one or more of the probe arrays to pass through a portion of the cooling surface 216. Alternative variations of the device include probes that pass through a portion of the cooling device.

FIG. 14B also shows a variation of the device having additional energy transfer elements 105 located in the cooling surface 216. As noted above, these energy transfer elements can include sources of radiant energy that can be applied either prior to the cooling surface contacting the skin, during energy treatment or cooling, or after energy treatment

FIG. 14C shows an aspect for use with methods and devices of the invention that allows marking of the treatment site. As shown, the cartridge 100 may include one or more marking lumens 226, 230 that are coupled to a marking ink 98. During use, a medical practitioner may be unable to see areas once treated. The use of marking allows the practitioner to place a mark at the treatment location to avoid excessive treatments. As shown, a marking lumen 226 may be placed proximate to the probe 104. Alternatively, or in combination, marking may occur at or near the cooling surface 216 since the cooling surface is directly above the treated region of tissue. The marking lumens may be combined with or replaced by marking pads. Furthermore, any type of medically approved dye may be used to mark. Alternatively, the dye may comprise a substance that is visible under certain wavelengths of light. Naturally, such a feature permits marking and visualization by the practitioner given illumination by the proper light source but prevents the patient from seeing the dye subsequent to the treatment.

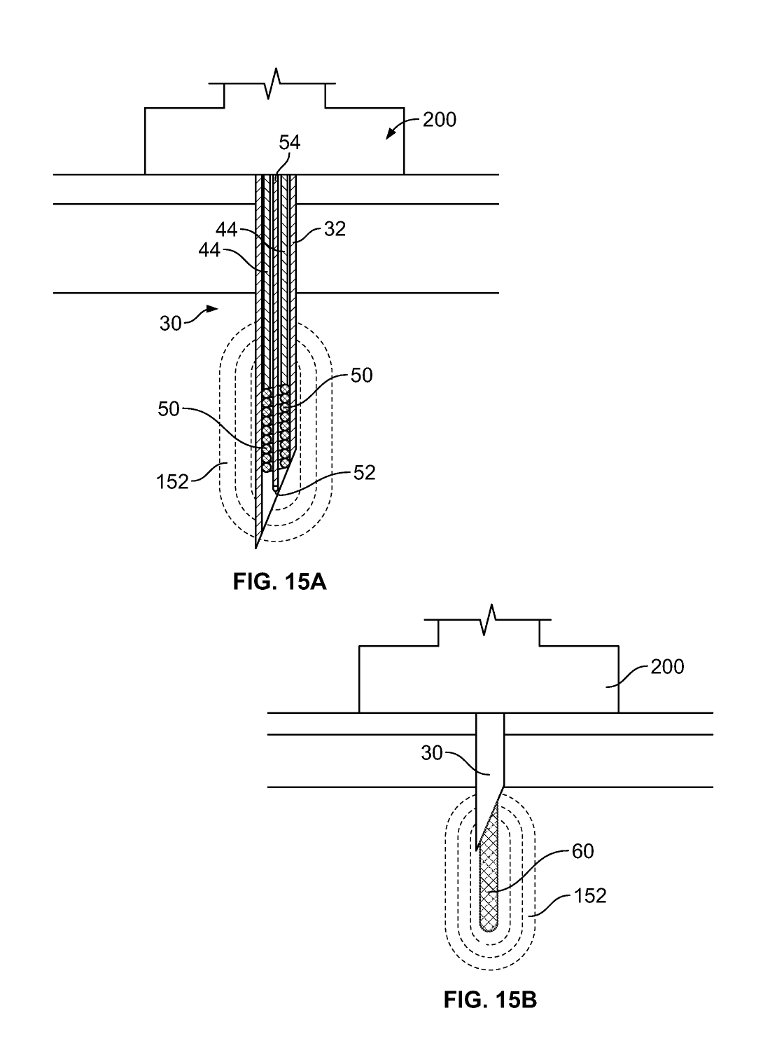

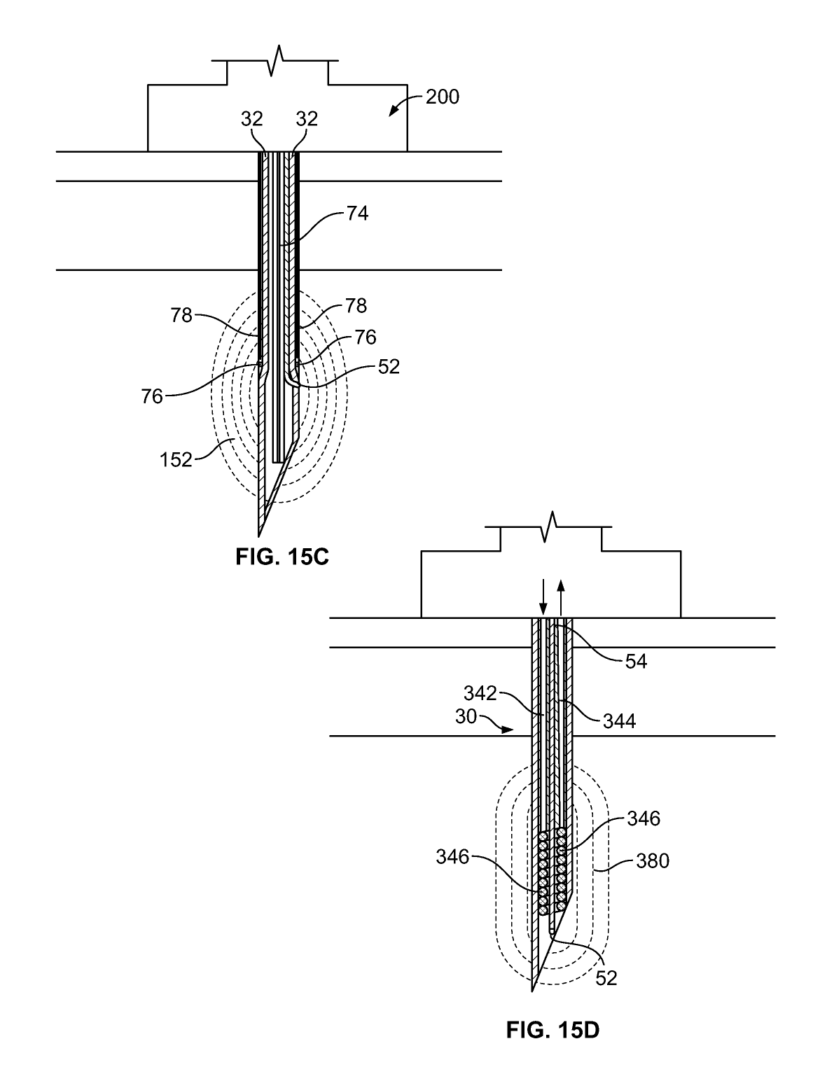

FIG. 15A shows an alternative variation of a probe that includes a resistive heating element 50 to supply therapeutic treatment to the tissue. The resistive heater 50 can be made of any number of typical nickel chrome alloys that produce thermal heat via electrical resistance. The heat produced by the heater 50 conducts through the probe walls 32 and into the dermal tissue. A temperature sensor 52 can be positioned anywhere as shown herein. However, in the illustrated variation, the sensor 52 is placed on the outer surface of the probe 30. This sensor 52 can provide temperature feedback to the system to adjust power delivery to the resistive heater 50 for producing desired energy delivery to the targeted dermal tissue 152.

FIG. 15B shows an alternative probe configuration. In this embodiment an energy element 60 advances out of the probe 30. The energy element 60 can be a resistive heater, an RF electrode, a cryoprobe, or any energy modality discussed herein were direct contact with the target tissue is beneficial. This variation allows the energy delivery element 60 to more directly contact the target tissue without having to transfer energy through the probe wall 32. Accordingly, this design allows for use of lower energy levels to achieve the same therapeutic effect. In those therapies where the tissue is heated, the targeted temperature can be reached in a shorter time period given the direct contact. In addition, the variation of FIG. 15B can employ a temperature sensor 52 as shown above.

FIG. 15C shows an additional variation of a probe 30 configuration. This variation contains a coaxial central conductor 74 and an outer conductor 78. It also contains insulators 76 that create a dipole for directing electrical energy in the microwave spectrum from the device into the tissue to heat the tissue 152. This microwave heater can also be used to treat dermal tissue and can rely on a temperature sensor 52 to adjust delivered power. In an alternate variation, the probe 30 can include shielding to direct the microwave energy in a particular direction to create a zone of treatment as described above.

FIG. 15D shows a variation of a cryogenic probe device. Typically, the device will produce a hypothermia effect within tissue. In one configuration, the probe 30 includes a delivery lumen 342 and return lumen 344 and a coiled heat exchanger 346. Cooled liquid or gas can be delivered through the delivery lumen 342 to the coiled heat exchanger 346 where it will cool the surrounding target tissue before exiting the probe 30 through the return lumen 344. The fluid or gas delivery can be controlled by measuring the target tissue temperature with temperature sensor 52 that is coupled to a control source (not shown) via conducting wires 54.

Clearly, any number of different cooling devices can be incorporated into the probe to produce a percutaneous hypothermia effect within tissue. For example, a percutaneous hypothermia treatment device can include a thermal electric cooler, TEC, such as a peltier device. Electric current can be delivered to the TEC to reduce its temperature such that it will cool the surrounding target tissue. The efficiency of the TEC can be optionally improved by providing a cooling device to remove heat generated by the side of the TEC that is not in contact with the target tissue. This cooling device can rely on the flow of a fluid or gas on the side of the TEC not in contact with the target tissue, or through a beat exchanger which is attached to the side of the TEC not in contact with the target tissue.

In any of the above variation, the energy sources can be configured as directional energy sources via the use of the appropriate insulation to direct energy to produce the treatment zones as described above.

Although the systems described herein may be used by themselves, the invention includes the methods and devices described above in combination with substances such as moisturizers, ointments, etc. that increase the resistivity of the epidermis. Accordingly, prior to the treatment, the medical practitioner can prepare the patient by increasing the resistivity of the epidermis. During the treatment, because of the increased resistivity of the epidermis, energy would tend to flow in the dermis.

In addition, such substances can be combined with various other energy delivery modalities to provide enhanced collagen production in the targeted tissue or other affects as described herein.

In one example, 5-aminolevulinic acid (ALA) or other photolabile compounds that generate a biologically active agent when present in the skin upon exposure to sunlight or other applied spectrums of activating light. Coatings or ointments can also be applied to the skin surface in order to stabilize the soft tissue. Temporarily firming or stabilizing the skin surface will reduce skin compliance and facilitate the insertions of the probes of the current device. An agent such as cyanoacrylate, spirit gum, latex, a facial mask or other substance that cures into a rigid or semi-rigid layer can be used to temporarily stabilize the skin. The topical ointments or coatings can be applied to enhance collagen production or to stabilize the skin for ease of probe insertion or both. Furthermore, topical agents can be applied to alter the electrical properties of the skin. Applying an agent which increases the impedance of the epidermal layer will reduce the conductance of RF current through that layer and enhance the conductance in the preferred dermal layer. A topical agent that penetrates the epidermal layer and is absorbed by the dermal layer can be applied that lowers the impedance of the dermal layer, again to enhance the conduction of RF current in the dermal layer. A topical agent that combines both of these properties to affect both the dermal and epidermal layers conductance can also be used in combination with RF energy delivery.

In addition to topical agents, the invention with its use of penetrating devices lends itself to the delivery of agents and materials directly to a specific region of tissue. For example, anesthetic agents such as lidocaine can be delivered through the probe to the dermis and epidermis to deaden nerve endings prior to the delivery of therapeutic energy. Collagen or other filler material can be delivered prior to, during or after energy delivery. Botulinum Toxin Type A, Botox, or a similar neurotoxin can be delivered below the skin layer to create temporary paralysis of the facial muscles after energy delivery. This maybe provide a significant improvement in the treatment results as the muscles would not create creases or wrinkles in the skin while the thermally treated collagen structure remodeled and collagenesis occurs.

Another means to enhance the tissue's therapeutic response is the use of mechanical energy through massage. Such an application of mechanical energy can be combined with the methods and systems described herein. Previously, devices have used massaging techniques to treat adipose tissue. For example, U.S. Pat. No. 5,961,475 discloses a massaging device that applies negative pressure as well as massage to the skin. Massage both increases blood circulation to the tissue and breaks done connections between the adipose and surrounding tissue. For example, these effects combined with energy treatment of the tissue to enhance the removal of fat cells.

The above variations are intended to demonstrate the various examples of embodiments of the methods and devices of the invention. It is understood that the embodiments described above may be combined or the aspects of the embodiments may be combined in the claims.

What is claimed is:

1. A method for applying energy treatment to a region of tissue beneath the epidermis, the method comprising:
positioning at least a portion of at least one probe beneath the epidermis, where the probe comprises a body having an outer perimeter; and
applying energy from the probe to create a zone of treatment, such that the exposure of energy to treat tissue is non-uniform about the outer perimeter of the probe and greatest in the zone of treatment.

2. The method of claim 1, where applying energy comprises applying an amount of energy to cause a therapeutic effect only in tissue within the zone of treatment.

3. The method of claim 1, further comprising rotating the probe to permit energy to tissue located about the outer perimeter of the probe.

4. The method of claim 1, wherein the probe includes at least one energy delivery element located within a passageway of the probe.

5. The method of claim 4, wherein the energy delivery element comprises an element selected from the group consisting of an acoustic transducer, an illumination source, a microwave energy supply, a resistive heat source, an RF energy probe, and a cooling source.

6. The method of claim 4, further comprising articulating the energy delivery element to change an angular position of a selective direction of energy delivery.

7. The method of claim 6, where the energy delivery element comprises an illumination source and a mirror, and where changing the angular position comprises repositioning the mirror.

8. The method of claim 1, further comprising measuring temperature beneath the epidermis and adjacent to the tissue receiving energy from the probe with a temperature sensor.

9. The method of claim 8, further comprising advancing the temperature sensor from the probe and into the tissue.

10. The method of claim 8, further comprising advancing the temperature sensor into a path of the energy.

11. The method of claim 1, wherein the probe comprises an opening within the outer perimeter such that the opening permits application of energy in the selective direction.

12. The method of claim 1, further comprising placing a plurality of probes beneath the epidermis.

13. The method of claim 12, further comprising placing at least two probes beneath the epidermis such that the respective zones of treatment of at least two probes intersect.

14. The method of claim 13, further comprising placing the plurality of probes in a circular pattern such that the respective zones of treatment of the probes intersect.

15. The method of claim 1, where positioning at least one probe beneath the epidermis comprises positioning the zone of treatment within dermal tissue.

16. The method of claim 1, where positioning at least one probe beneath the epidermis comprises positioning the zone of treatment within a layer of subcutaneous fat.

17. The method of claim 1, further comprising placing a tissue engaging surface against an epidermal layer of tissue, and advancing the probe through the epidermis to position the probe beneath the epidermis.

18. The method of claim 17, where advancing the probe comprises advancing the probe at an oblique angle relative to the tissue engaging surface.

19. The method of claim 1, wherein the energy causes heating of the tissue.

20. The method of claim 1, wherein the energy causes cooling of the tissue.

21. A medical device for delivering energy from a power supply to tissue, the medical device comprising:
a body having a tissue engaging surface;
at least one probe extending from the tissue engaging surface, having a tip adapted to penetrate tissue, and where a sidewall of the probe comprises an opening;
an energy delivery element coupleable to the power supply and positioned within the probe such that energy transmitted by the energy delivery element passes through the opening of the sidewall to treat tissue.

22. The medical device of claim 21, where the energy delivery element is rotatable.

23. The medical device of claim 21, where the probe is rotatable.

24. The medical device of claim 21, where the energy delivery element is configured to produce sufficient energy through the opening to create a zone of treatment in the tissue.

25. The medical device of claim 21, wherein the energy delivery element comprises an element selected from the group consisting of an acoustic transducer, an illumination source, a microwave energy supply, a resistive heat source, an RF energy probe, a cooling source.

26. The medical device of claim 21, where a portion of the energy delivery element is pivotable to allow for a change in an angular position of energy passing through the opening.

27. The medical device of claim 26, where the energy delivery element comprises an illumination source and a mirror, and wherein the mirror is adapted to be repositioned to change the angular position of the energy.

28. The medical device of claim 21, further comprising a temperature sensor located within the probe and proximate to the opening.

29. The medical device of claim 21, further comprising a temperature sensor located within the probe and advanceable from the probe.

30. The medical device of claim 29, where the temperature sensor is adapted to be advanced adjacent to the opening.

31. The medical device of claim 21, wherein the probe comprises a covering member over the opening.

32. The medical device of claim 21, where the at least one probe comprises at least a pair of probes having openings aligned such that energy from each respective energy delivery element treats the same region of tissue.

33. The medical device of claim 21, where the at least one probe comprises at least two rows of probes.

34. The medical device of claim 21, where the at least one probe comprises a plurality of probes arranged in a circular pattern.

35. The medical device of claim 21, where the at least one probe forms an oblique angle relative to the tissue engaging surface.

36. The medical device of claim 21, where the at least one probe is advanceable from the tissue engaging surface to form an oblique angle relative to the tissue engaging surface.

37. The medical device of claim 21, wherein the energy delivery element is adapted to heat tissue.

38. The medical device of claim 21, wherein the energy delivery element is adapted to cool tissue.

39. A method for applying energy treatment to a region of tissue beneath the epidermis, the method comprising:
positioning at least one probe beneath the epidermis, where the probe comprises an outer perimeter and at least one opening in a sidewall; and
delivering a pressurized fluid through the sidewall to mechanically disrupt a region of tissue adjacent to the opening.
